(19) United States
(12) Patent Application Publication
(10) Pub. No.: US 2024/0254567 A1
(43) Pub. Date: Aug. 1, 2024

(54) DIAMOND MAGNETOMETRY DETECTION OF BIOLOGICAL TARGETS

(71) Applicant: COLUMBUS NANOWORKS, INC., Columbus, OH (US)

(72) Inventors: Arfaan RAMPERSAUD, Columbus, OH (US); Isaac RAMPERSAUD, Columbus, OH (US); Charles FLECTCHER, Columbus, OH (US); Mia FORREN, Columbus, OH (US)

(73) Assignee: COLUMBUS NANOWORKS, INC., Columbus, OH (US)

(21) Appl. No.: 18/636,061

(22) Filed: Apr. 15, 2024

Related U.S. Application Data
(63) Continuation-in-part of application No. PCT/US22/46904, filed on Oct. 17, 2022.
(60) Provisional application No. 63/256,093, filed on Oct. 15, 2021.
(60) Provisional application No. 63/549,890, filed on Feb. 5, 2024.

(51) Int. Cl. C12Q 1/6886; C12Q 1/6818

(52) U.S. Cl. CPC C12Q 1/6886; C12Q 1/6818; C12Q 2600/178

(57) ABSTRACT

A composition includes a fluorescent nitrogen-vacancy nanodiamond conjugated to a probe. The probe specifically binds to a target material when present in a biological sample. A device includes a flow cell containing a plurality of fluorescent nitrogen-vacancy nanodiamonds immobilized on a surface of the flow cell. Each of the plurality of fluorescent nitrogen-vacancy nanodiamonds is conjugated to a probe. A diamond magnetometry system includes an ODMR measurement system, a spin-lattice relaxation time (T1) system, a spin-spin relaxation time (T2) measurement system, and a flow cell.

### REFERENCE TO RELATED APPLICATIONS

This application is a continuation-in-part of International Application No. PCT/US22/46904, filed Oct. 17, 2022, which claims priority to and the benefit of U.S. Provisional Application No. 63/256,093 filed Oct. 15, 2021, both of which are hereby incorporated by reference in their entirety. This application also claims priority to and the benefit of U.S. Provisional Application No. 63/549,890 filed Feb. 5, 2024, which is hereby incorporated by reference in its entirety.

### FIELD

The present disclosure is directed to compositions, devices, and systems that employ diamond magnetometry to provide improved detection of specific target materials in samples. More specifically, the present disclosure is directed to compositions, devices, and systems that detect target biological materials in biological samples.

### BACKGROUND

Specific target materials in biological samples can help diagnose diseases by detection of their presence, absence, or concentration. These target materials include, but are not limited to, nucleic acids (including oligonucleotides), proteins (including oligopeptides), electrolytes, and other substances that result from, or are increased or depleted due to, disease.

Regarding breast cancer as a target, it is known that breast cancer is the second most common cause of cancer associated mortality in women in the United States. While there has been substantial progress in the treatment of breast cancer patients, early detection is essential for the best therapeutic outcome. Mammography is a well-established imaging technique for breast cancer screening, and combining this with a complementary method, such as liquid biopsy of blood or aspirate or other bio fluid from a clinical subject, can provide a more comprehensive understanding of a patient's cancer status. Liquid biopsies are non-invasive blood tests that detect signs of cancer, based on the analysis of protein and nucleic acid biomarkers. They also provide measurable information about disease progression, recurrence, and relapse. Of course, the use of liquid biopsies is not limited to breast or any other cancer and can be used to detect any of a variety of targets.

Detection and measurement of target materials can also track patient response to treatment and facilitate high throughput screening to evaluate efficacy of potential treatments for such diseases by measuring changes in levels of the target.

Due to generally having the lowest time and sample size requirements, the most widely employed methods for detection of nucleotide target materials, including deoxyribonucleic acid (DNA) and ribonucleic acid (RNA), are polymerase chain reaction (PCR)-based methods, such as, for example, Beads, Emulsion, Amplification, Magnetics (BEAMing) and droplet digital PCR (ddPCR), which have limits of detection of 0.01 and 0.001%, respectively.

PCR amplifies pieces of nucleotide target materials in a sample, for example, DNA, by several orders of magnitude to improve detection. A PCR procedure typically requires about 25 to 40 temperature cycles, with three 2-minute steps required per cycle, for a total of about 2.5 to 4 hours.

For detection of specific proteins, the most widely used tests are spectrometry-based and antibody-based methods. These methods require several hours to complete and require equipment that is not considered portable.

The ability to detect biological and chemical targets with single molecule sensitivity, in real time, would radically advance the ability to monitor biomarkers of human health and performance, as well as rapidly sense chemical, and biological targets/threats. Such a sensor has not been achieved to date mainly due to the technical limitations of current technologies. Furthermore, technologies such as polymerase chain reaction (PCR), next-generation sequencing, BEAMing, digital PCR (dPCR), and loop-mediated isothermal amplification (LAMP) require some form of amplification of target molecules and require additional components and processes that reduce speed and increase cost.

Generally, the existing technologies are relatively slow, creating practical timing challenges to rapidly diagnose a disease state and to rapidly and efficiently screen for treatment response. Compositions, devices, and systems not suffering from these drawbacks are desirable in the art.

### SUMMARY

Provided are systems, compositions, reagents, processes, and devices for detection of target materials in biological samples employing diamond magnetometry.

Embodiments of the present disclosure, for example, in comparison to concepts failing to include one or more of the features disclosed herein, provide decreased detection time requirements, lower cost, equivalent or lower limits of detection, or combinations thereof.

In a first embodiment, provided is a system for detecting a target material, comprising: a plurality of fluorescent nitrogen-vacancy nanodiamond-probe-reporter conjugates (MND-probe conjugates), each MND-probe conjugate comprising: at least one fluorescent nitrogen-vacancy nanodiamond (FND); at least one reporter selected from the group consisting of a magnetic particle (MP), a paramagnetic complex, and a small molecule (for example, biotin or other metabolite, or a small peptide); and at least one polymeric probe comprising a biomolecule selected from the group consisting of at least one polynucleotide, at least one polypeptide, and a combination thereof, wherein the polymeric probe is characterized as having binding specificity to the target material; wherein each of the at least one FND and the at least one reporter is linked to the at least one polymeric probe, and wherein a distance between the FND and the reporter in the MND-probe conjugate changes when the at least one polymeric probe binds to the target material and displaces or releases the target material.

In some particular embodiments, the at least one reporter is selected from the group consisting of a nanoscale MP, a paramagnetic complex, a gadolinium complex, and a chemical.

In some particular embodiments, the at least one reporter is a chemical comprising biotin, the system also comprising a magnetic particle comprising avidin or streptavadin.

In some particular embodiments, the biomolecule is at least one polynucleotide, the at least one polynucleotide being selected from the group consisting of duplex deoxyribonucleic acid (DNA) comprising a first DNA strand and a second DNA strand, at least a portion of the second DNA strand being complementary with at least a portion of the first DNA strand; a pair of polynucleotide strands comprising a first strand and an aptamer strand, the aptamer strand being complementary to at least a portion of the first strand; and a single polynucleotide strand comprising two regions of complementarity.

In some particular embodiments, each of the plurality of MND-probe conjugates includes one FND, one reporter, and one polymeric probe.

In some particular embodiments, the target material is selected from the group consisting of (i) a small ribonucleic acid (RNA) of less than 200 bases comprising a microRNA (miR), (ii) a small ribonucleic acid (RNA) of less than 200 bases comprising a small noncoding RNA (sncRNA), (iii) a small ribonucleic acid (RNA) of less than 200 bases comprising a transfer RNA (tRNA), (iv) a small ribonucleic acid (RNA) of less than 200 bases comprising a short interfering RNA (siRNA), (v) a deoxyribonucleic acid duplex, (vi) a peptide, (vii) a small molecule, and (viii) combinations thereof.

In some particular embodiments, the polymeric probe comprises a nucleic acid duplex comprising a first nucleic acid strand coupled to the reporter and a second nucleic acid strand coupled to the FND, the second nucleic acid strand being at least partially complementary to the first nucleic strand, each of the first and second strands having a first end and a second end corresponding to a first end and a second end of the polymeric probe; at least one of the nucleic acid strands has binding affinity for the target material; the first nucleic acid strand is four to eight bases shorter than the second nucleic acid strand; the second nucleic acids comprise four to eight bases that are complementary to the first nucleic acid and form a toe hold region when the first and second nucleic acid strands are hybridized; and each of the reporter and the FND are coupled to the respective first and second nucleic acid strands at the same end of the MND-probe conjugate or at opposite ends of the MND-probe conjugate.

In some particular embodiments, the at least one FND is coated with a first plurality of chemical functional group linkers comprising glycidols and the at least one reporter is coated with a second plurality of chemical functional group linkers comprising carboxyl moieties to chemically couple the FND to the reporter, and wherein the at least one FND has a particle size in the range of about 10 nm to about 1000 and the at least one reporter has a magnetic core size in the range of about 5 nm to about 100 nm.

In some particular embodiments, the polymeric probe is characterized as having binding specificity to the target material, wherein an FND-probe conjugate binds to the target material, and wherein a distance between the FND and the reporter in the MND-probe conjugate changes when the FND-probe conjugate binds to the target material.

In some particular embodiments, the polymeric probe is characterized as having binding specificity to the target material, wherein a reporter-probe conjugate binds to the target material, and wherein a distance between the FND and the reporter in the MND-probe conjugate changes when the reporter-probe conjugate binds to the target material.

In another embodiment, provided is a process for detecting the presence of a target material in a sample, the process comprising: providing the plurality of MND-probe conjugates according to claim 1; providing the sample; providing an interrogation system capable of detecting at least one measurable change relating to the displacement of the reporter toward or away from the FND; introducing the plurality of MND-probe conjugates into the detection system in contact with the sample suspected of containing the target material; and interrogating the MND-probe conjugates-sample combination to detect a measurable change.

In some particular embodiments, the measurable change is a change in fluorescence.

In some particular embodiments, the process further comprises providing a flow cell and a fluid medium for receiving the sample, the sample comprising a biological sample from one of cell, tissue, or a combination thereof, wherein one or more of the plurality of MND-probe conjugates are immobilized on a transparent substrate of the flow cell and the flow cell is configured to flow the sample over the immobilized reagents.

In some particular embodiments, the transparent substrate is selected from the group consisting of a glass coverslip, a glass slide, a glass plate, an array titration tray, and a clear polymeric substrate.

In some particular embodiments, the process further comprises providing a transparent substrate and a fluid medium for receiving the sample, the sample comprising a biological sample from one of cell, tissue, or a combination thereof, wherein one or more of the plurality of MND-probe conjugates are immobilized by drop casting on the transparent substrate with an immobilizing agent sufficient to provide binding capture for a diamond concentration in the range of about 0.1 ng/L to about 1000 ng/mL and a time period in the range of about 10 seconds to about 10 hours such hat the distribution of nanodiamonds is a punctate single layer across the substrate surface.

In some particular embodiments, the interrogation system comprising optics configured to generate and measure the polarization of an NV-center of the FND or one or more of optically detected magnetic resonance (ODMR), a spin-lattice relaxation time (T1), a spin-spin relaxation time (T2) of the plurality of magnetic particles, or a combination thereof.

In some particular embodiments, measuring ODMR of the plurality of fluorescent nitrogen-vacancy nanodiamonds after exposure to the biological sample determines a presence or an absence of the target material in the biological sample based on the polarization, the ODMR, the T1, or the T2, and wherein measuring a spin-lattice relaxation time (T1) or a spin-spin relaxation time (T2) of the plurality of MND-probe conjugates after exposure to the biological sample determines a presence or an absence of the target material in the biological sample based on values of T1 or T2.

In some particular embodiments, the process is used in a breast cancer screening.

In another embodiment, provided is a reagent for detection of a target material, the reagent comprising: a plurality of fluorescent nitrogen-vacancy nanodiamond-probe-reporter conjugates (MND-probe conjugates), each MND-probe conjugate comprising: at least one fluorescent nitrogen-vacancy nanodiamond (FND); at least one reporter comprising a magnetic nanoparticle particle (MNP); and at least one polymeric probe, the polymeric probe comprising a first nucleic acid strand coupled to the MP and a second nucleic acid strand coupled to the FND, the first nucleic acid strand being at least partially complementary to the second nucleic acid strand to releasably couple the MP to the FND via the interaction between the complementary first and second nucleic acid strands, one of the nucleic acid strands having binding affinity for the target material; wherein a distance between the FND and the reporter in the MND-probe conjugate changes when the at least one polymeric probe binds to the target material and displaces or releases the target material.

In some particular embodiments, the target material is cortisol and the cortisol causes a conformational change in the polymeric probe which produces positional changes in a spatial relationship of the MNP and the FND in the MND-probe conjugate, or wherein the target material is a microribonucleic acid biomarker of breast cancer and the polymeric probe comprises a deoxyribonucleic acid having a single-stranded toehold portion for the target material to bind and initiate strand displacement of the polymeric probe.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

FIG. 1 schematically shows optics for ODMR of a diamond magnetometry device in an embodiment of the present disclosure.

FIG. 2 schematically shows a flow cell for the diamond magnetometry device of FIG. 1.

FIG. 3 schematically shows expected outcomes for ODMR in the diamond magnetometry device of FIG. 1.

FIG. 4 schematically shows expected outcomes for spin-lattice relaxation time (T1) and spin-spin relaxation time (T2) in a diamond magnetometry device.

FIG. 5A schematically shows a magnetic particle (MP) as a reporter held near a nitrogen-vacancy (NV) diamond surface by attaching either an NV diamond or MP to the intein and the other to the extein.

FIG. 5B schematically shows an NV diamond attached to one extein bound to an intein fragment and the MP to the other.

FIG. 5C schematically shows detection in an intein/extein system via cis-splicing.

FIG. 6A schematically shows antibody-antigen-antibody binding to bring the NV and MP together.

FIG. 6B schematically shows detecting antibody-secondary antibody binding to bring the NV and MP together.

FIG. 7 schematically shows a process for forming a fluorescent nitrogen-vacancy nanodiamond (FND)-probe-magnetic nanoparticle (MP) conjugate, also referred to as a “MND-probe conjugate” comprising and least one FND and at least one MP.

FIG. 8A shows ODMR of a thiolated FND.

FIG. 8B shows ODMR of an MND-probe conjugate.

FIG. 9A shows a streptavidin (SA)-biotin binding system.

FIG. 9B shows a fluorescence heat map of the FNDs for the binding system of FIG. 9A.

FIG. 9C shows the ODMR spectra for the binding system of FIG. 9A.

FIG. 10 shows ODMR of an FND-DNA strand conjugate and an DNA-MP conjugate.

FIG. 11 schematically shows a process for forming an MND-probe conjugate stem-loop nucleic acid conjugate.

FIG. 12 shows ODMR of an MND-probe conjugate stem-loop nucleic acid conjugate in the presence and in the absence of a target material.

FIG. 13 shows schematically a DM process with magnetic nanoparticles and fluorescent NV-center nanodiamond linked together through a short duplex DNA chain.

FIG. 14 shows a bar chart of detection results from an ODMR process shown in FIG. 13.

FIG. 15A shows a log-log plot of the difference in contrast between a fluorescent nanodiamond and a fluorescent nanodiamond linked to a magnetic nanoparticle held together through a short duplex DNA.

FIG. 15A shows a log-log plot of the difference in contrast between an NV-center nanodiamond magnetic nanoparticle, linked together through a short duplex DNA chain, before and after displacement of the magnetic nanoparticle DNA

FIG. 16 shows the specificity of detection of miR-21 oligonucleotide over other miRNA oligonucleotides.

FIG. 17 shows the alternative analysis in an aptamer probe conjugate system.

FIG. 18A schematically shows a positive result for a first MND-probe conjugate system.

FIG. 18B schematically shows a negative result for the MND-probe conjugate system of FIG. 18A.

FIG. 19A schematically shows a positive result for a second MND-probe conjugate system.

FIG. 19B schematically shows a negative result for the MND-probe conjugate system of FIG. 19A.

FIG. 20A schematically shows a positive result for a third MND-probe conjugate system.

FIG. 20B schematically shows a negative result for the MND-probe conjugate system of FIG. 20A.

FIG. 21A schematically shows a positive result for a fourth MND-probe conjugate system.

FIG. 21B schematically shows a negative result for the MND-probe conjugate system of FIG. 21A.

Wherever possible, the same reference numbers will be used throughout the drawings to represent the same parts.

As depicted in one or more of the drawings and the specification hereof, the following polynucleotides are described herein as possible probes for use according to the disclosure. For the avoidance of doubt, these polynucleotides are examples of probes or probe conjugates (A-probe/aptamer, duplex, or stem loop forms), and are not in any way limiting of the scope of the embodiments of compositions, systems and methods hereof.

### Polynucleotides:

(SEQ ID NO: 1)
5′ GCCCGCATGTTCCATGGATAGTCTTGACTAGTCGTCCC 3′

Having a length of 38 bases, this is an aptamer for cortisol binding; synthetic polynucleotide (See Niu, C., Ding, Y., Zhang, C., & Liu, J. (2022). Comparing two cortisol aptamers for label-free fluorescent and colorimetric biosensors. Sensors & Diagnostics, 1(3), 541-549. URL//doi.org/10.1039/d2sd00042c, and K.-A. Yang, H. Chun, Y. Zhang, S. Pecic, N. Nakatsuka, A. M. Andrews, T. S. Worgall and M. N. Stojanovic, ACS Chem. Biol., 2017, 12, 3103-31.

(SEQ ID NO: 2)
5′ GGCGGTCAACATCAGTCTGATAAGCTACCTGACCGCC 3′

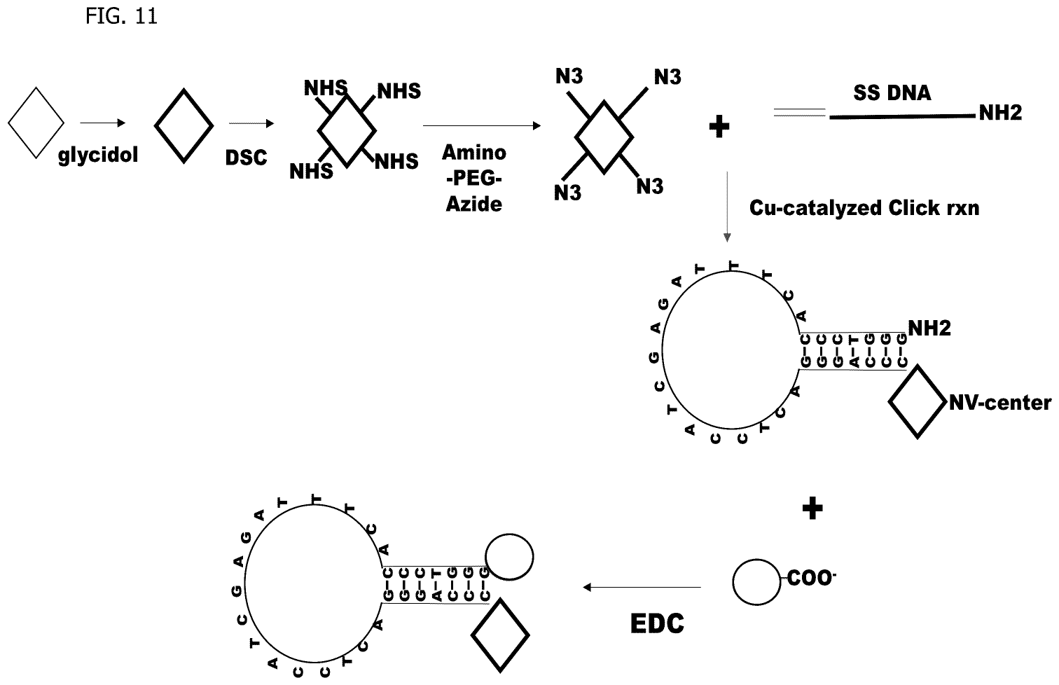

Having a length of 37 bases, this is a stem loop design for binding microRNA 21 (miR21); referencing FIG. 11, the underlined DNA sequence represents the stem region and is not naturally occurring; the 23-base pair (bp) region between the underlined sequences, corresponds to a single-strand region that is complementary to miR-21; Organism—Homo Sapiens, GenBank accession MI0000077; miRbase accession MIMAT0000076.

(SEQ ID NO: 3)
5′ GGGUAGCUUAUCAGACUGAUGUUGAC 3′

Referencing FIG. 11, (gggtagcttatcagactgatgttgac replaced U with T for generation of sequence listing) this is the RNA sequence for the human microRNA-21 (hsa-mir-21); Organism-Homo Sapiens; Entrez gene MIR21; miRbase accession MR0003024505

(SEQ ID NO: 4)
5′ CCCAGGGACTCCATCGAGATTTCACCCTGGG 3′

Having a length of 31 bases, this is a stem-loop DNA design for binding B-raf mRNA; referencing FIG. 11, the underlined DNA sequence represents the stem region and is not naturally occurring; the 17 base region between the underlined region hybridizes to the mRNA encoding B-raf protooncogene serine/threonine kinase (BRAF); Organism—Homo Sapiens; GenBank:OP680445.1

(SEQ ID NO: 5)
5′ ATGCTGTCTCACGCACATGGATGGTTTGGACAAATTTGATTCAAGT
CTGA 3′

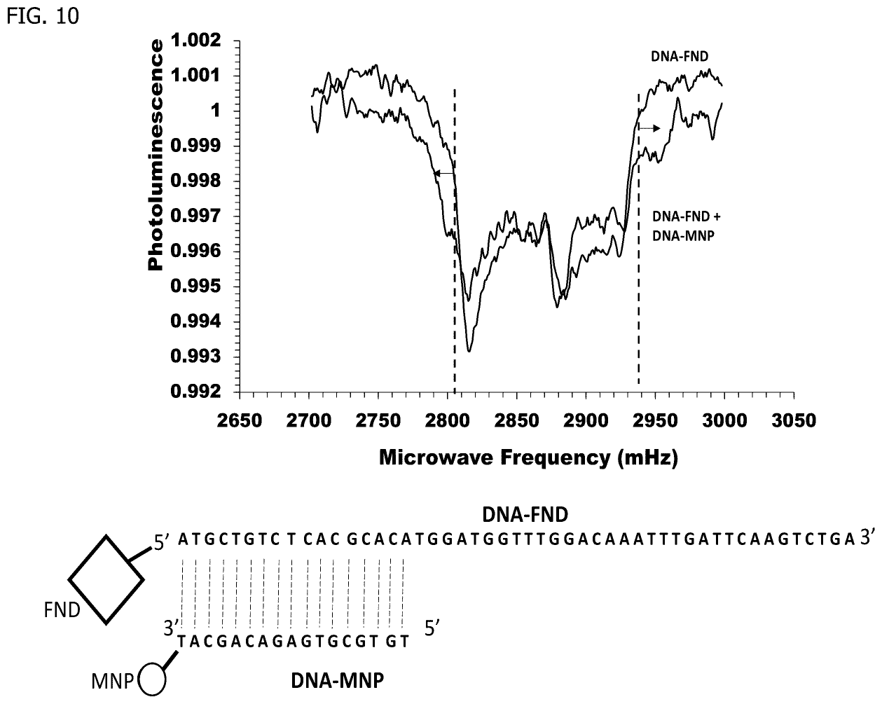

Referencing FIG. 10, this is a 50 base DNA sequence that was generated by the author using a random DNA sequence generator (http://www.faculty.ucr.edu/-mmaduro/random.htm).

(SEQ ID NO: 6)
5′ TGTGCGTGAGACAGCAT 3′

Referencing FIG. 10, this not naturally occurring DNA sequence hybridizes to the 5′ end of the 50 base DNA sequence.

(SEQ ID NO: 7)
5′-ATACCGAGAGCCCTGCTG-3′

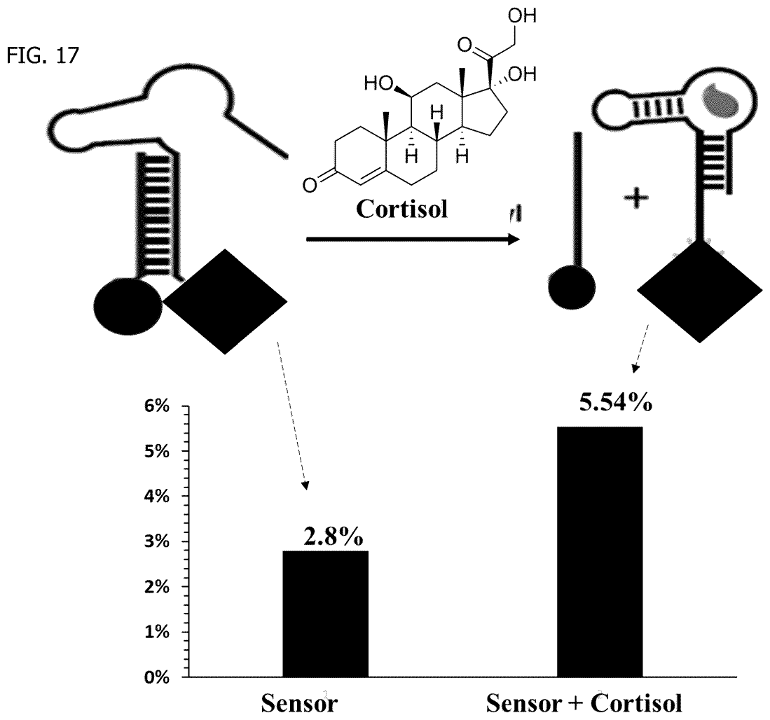

This sequence for the A-probe as depicted in FIG. 17 having complementary to the aptamer and binding affinity for the target, in this case, cortisol.

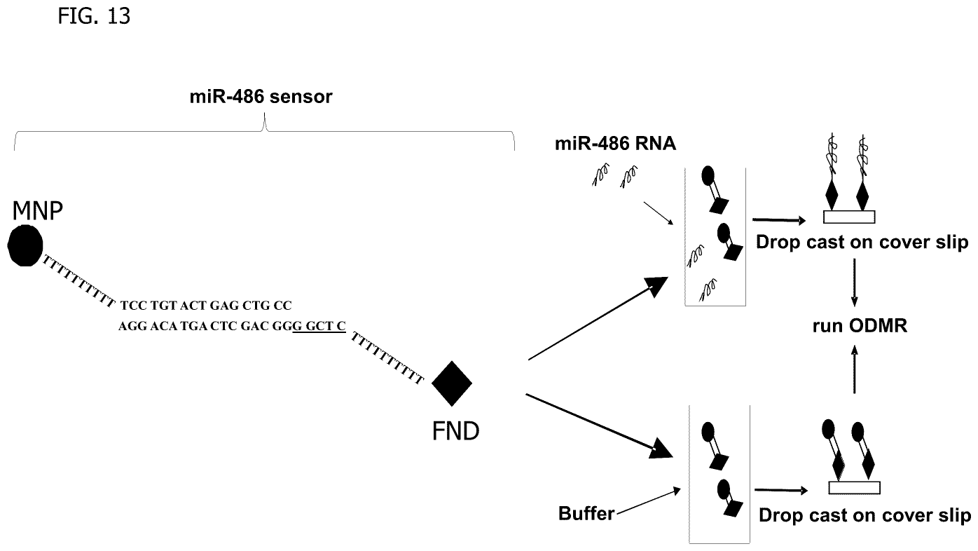

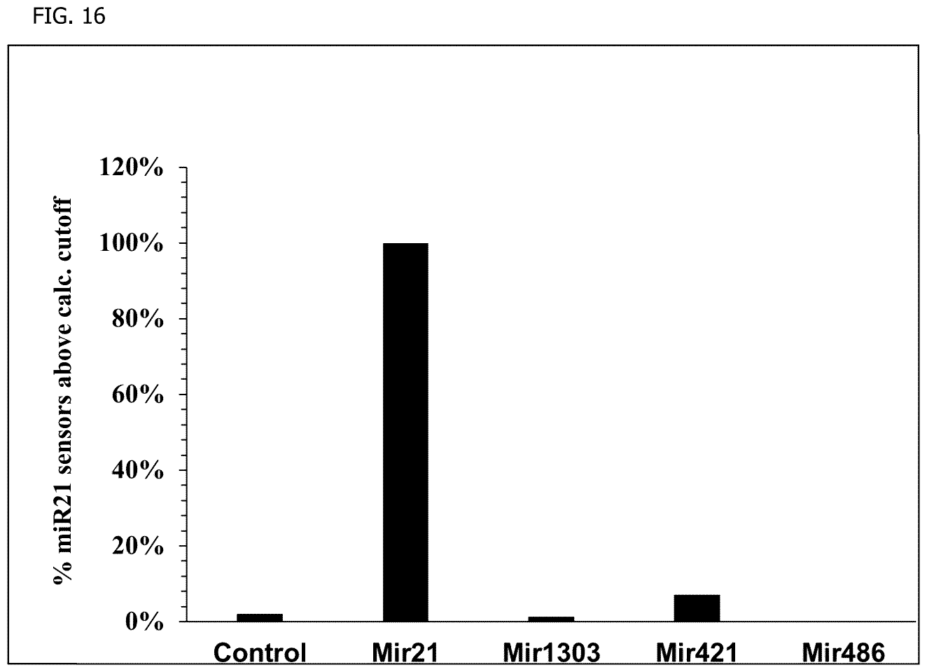

(hsa-miR-21)
(SEQ ID NO: 8)
5′-UAGCUUAUCAGACUGAUGUUGA-3′
(hsa-miR-486
(SEQ ID NO: 9)
5′-UCCUGUACUGAGCUGCCCCGAG-3′
(hsa-mir-421)
(SEQ ID NO: 10)
5′-AUCAACAGACAUUAAUUGGGCGC-3′
(hsa-let-7a)
(SEQ ID NO: 11)
5′-UGAGGUAGUAGGUUGUGUGGUU-3′
(hsa-miR-195)
(SEQ ID NO: 12)
5′-UAGCAGCACAGAAAUAUUGGC-3′
(hsa-miR-145)
(SEQ ID NO: 13)
5′-CUCUUGAGGGAAGCACUUUCUGU-3′
(hsa-miR-16)
(SEQ ID NO: 14)
5′-UAGCAGCACGUAAAUAUUGGCG-3′
(hsa-miR-34a)
(SEQ ID NO: 15)
5′-UGGCAGUGUCUUAGCUGGUUGU-3′
(hsa-miR-941)
(SEQ ID NO: 16)
5′-CACCCGGCUGUGUGCACAUGUGC--3′
(SEQ ID NO: 17)
5′-TCAACATCAGTCTGATAAGCTA-3′ (complementary DNA
sequence for hsa miR-21 in FIG. 16)
(SEQ ID NO: 18)
5′-CTCGGGGCAGCTCAGTACAGGA-3′ (complementary DNA
sequence for has miR-486 in FIG. 13)
(hsa mir-10b)
(SEQ ID NO: 19)
5′-UACCCUGUAGAACCGAAUUUGUG-3′
(hsa miR-155)
(SEQ ID NO: 20)
5′-UUAAUGCUAAUCGUGAUAGGGGUU-3′
(hsa miR-124)
(SEQ ID NO: 21)
5′-CGUGUUCACAGCGGACCUUGAU-3′
(hsa miR-181b)
(SEQ ID NO: 22)
5′-AACAUUCAUUGCUGUCGGUGGGU-3′

### DETAILED DESCRIPTION

In the case of in vitro diagnostics, there is a need for affordable point-of-care (POC) target detection that provides quicker test results with sensitivity that is equal to, or better than, that currently attainable from PCR, spectrometry, antibody-dependent assays (immunoassays), and other methods currently in use. In addition, there is a need for methods/technologies for rapid, high-throughput screening of drugs/genetic therapies for diseases that are detected by the above methods, as well as the means by which to better define therapeutic windows for such treatments.

According to the teachings of the instant disclosure, diamond magnetometry utilizes engineered diamonds, specifically, diamonds with NV centers (“NV center diamonds”), to detect changes in a magnetic field associated with at least one NV center diamond when there is a change in proximity between a reporter material (referred to herein as at least one “reporter”) and the at least one NV center diamond. According to some embodiments of the instant disclosure, the at least one NV center diamond is a nanoscale fluorescent NV center diamond, referred to herein as a “fluorescent nanodiamond” or an “FND.” The reporter may be a material that is magnetic or paramagnetic, or a material, for example, a metabolite, such as biotin, that binds to a reporter conjugate material that is magnetic or paramagnetic, for example avidin or streptavidin to which is coupled at least one magnetic or paramagnetic particle, as further described herein. A change in proximity between the at least one NV center diamond and the reporter results in a detectible change in the magnetic field around the FND based on the effects of the magnetic properties of the reporter on the probe. Thus, the at least one NV center diamond can be employed with at least one reporter in a variety of systems that enable the detection of a perturbation or change in the system that moves the at least one NV center diamond and the at least one reporter into or out of proximity of one another whereby the system operates as a sensor that can indirectly detect the change in the system based on the change in the magnetic field of the at least one NV center diamond.

According to the instant disclosure, in various embodiments, the inventors provide a system that includes at least one FND and at least one reporter and at least one probe to which the at least one FND and at least one reporter, respectively, are coupled such that a change in the conformation of the probe results in a detectible change in the magnetic field associated with the FND. In various embodiments, at least a portion of the probe has binding specificity for a target that is sought to be detected in a biological sample such that binding of the at least a portion of the probe to the target results in a change in the proximity of the at least one FND and the at least one reporter. As disclosed herein, the provided FND-probe-reporter conjugates (also referred to as “MND-probe conjugates”) are disclosed for use in biological systems using biomolecular probes.

It will be appreciated by one of ordinary skill that while many of the references herein are made to MND-probe conjugates which include a magnetic particle (MP), other reporters may be used and the examples and descriptions reference MPs are not limiting. It will further be appreciated that the compositions, systems and methods are not limited to probes as specifically exemplified herein below, and can include any probe that enables indirect detection of changes to the probe that alter the proximity of the FND and reporter which are detected using diamond magnetometry.

As further described herein, any of a variety of reporters and probes may be used. According to the disclosure, in some embodiments, the probe is a biomolecule, for example a polynucleotide construct comprising one, two or more strands of polynucleotide, each polynucleotide strand selected from ribonucleic acid and deoxyribonucleic acid, or a protein or peptide. In some particular embodiments the biomolecule is a polynucleotide duplex comprising complementary strands of polynucleotides wherein each of the reporter and the FND, respectively, are coupled to the respective polynucleotide strands. In some embodiments, the duplex DNA includes a toehold region, as described herein below. In some embodiments, complementary polynucleotide strands, for example, DNA, is in the form of an aptamer and A-probe complex wherein the A-probe is a short oligonucleotide with complementary to the aptamer and binding affinity for a target, as described herein below According to such embodiments, association and dissociation of the complementary polynucleotide strands results in a change in proximity between the FND and the reporter which in turn results in a change in the magnetic field around the FND that can be detected by various means, as disclosed herein. In some particular embodiments, the reporter is itself not magnetic or paramagnetic, and the system further includes a reporter conjugate wherein an association between the reporter and the reporter conjugate (for example biotin as the reporter and avidin or streptavidin tagged with a magnetic or paramagnetic particle, as the reporter conjugate) provides the detectible effect on the FND magnetic field. Such embodiments are further described herein.

In an exemplary clinical application according to the disclosure, the inventive compositions, systems and methods may be employed to detect the presence of breast cancer based on target polynucleotide molecules known to be associated with certain breast cancers. Of course, one of ordinary skill in the art will realize that the compositions, systems and methods may be employed to detect any of a wide variety of targets, wherein the presence of a target is associated with the presence of a disease state, such as cancer, or a contaminant, infectious agent, or toxin, or other target of biological relevance. In accordance with various embodiments, provided are compositions/reagents, systems, devices, and methods for detecting a target material in a sample. In some exemplary embodiments, the sample is a biological sample taken from an organism, cell culture or other material that may include biological material from an organism. In some embodiments, the target material is a biomaterial, such as a polynucleotide (double or single stranded), a polypeptide (a protein or oligopeptides) or a combination thereof. In various embodiments, one or more reagents are used to detect the binding (typically non covalent) between the target material in a sample and a probe that has affinity for the target material wherein the systems include probes that are bound with one or both FNDs and MPs such that binding between the target and the probe results in a change of relative distance between the FND and MP as bound to the probe to produce a detectable change according to the interrogation and detection methods disclosed herein.

In exemplary embodiments, diamond magnetometry detection of target DNA, RNA, proteins, and/or other specific target materials in samples, including, but not limited to, biological samples, can reduce or eliminate the need for DNA amplification, immunoassays, and spectrometric identification, thereby reducing the time required for target detection from hours to minutes. In exemplary embodiments, diamond magnetometry provides sensitivity equal to that of the alternative methods at a lower cost.

A composition according to the instant disclosure includes a fluorescent nitrogen-vacancy nanodiamond, or FND, conjugated to at least one probe that includes at least one reporter and one or more features specific for recognizing and binding to a target material in a test sample. In a first embodiment, provided is a system for detecting a target material, comprising: a plurality of fluorescent nitrogen-vacancy nanodiamond-probe-reporter conjugates (MND-probe conjugates), each MND-probe conjugate comprising: at least one fluorescent nitrogen-vacancy nanodiamond (FND); at least one reporter selected from the group consisting of a magnetic particle (MP), a paramagnetic complex, and a small molecule (for example, biotin or other metabolite, or a small peptide); and at least one polymeric probe comprising a biomolecule selected from the group consisting of at least one polynucleotide, at least one polypeptide, and a combination thereof, wherein the polymeric probe is characterized as having binding specificity to the target material; wherein each of the at least one FND and the at least one reporter is linked to the at least one polymeric probe, and wherein a distance between the FND and the reporter in the MND-probe conjugate changes when the at least one polymeric probe binds to the target material and displaces or releases the target material.

A device may include a flow cell containing a plurality of fluorescent nitrogen-vacancy nanodiamonds immobilized on a surface of the flow cell. Each of the plurality of fluorescent nitrogen-vacancy nanodiamonds is conjugated to a probe. The probe specifically binds to a target material when present in a biological sample. A diamond magnetometry system includes one or more of an optically detected magnetic resonance (ODMR) measurement system, a spin-lattice relaxation time (T1) system, a spin-spin relaxation time (T2) measurement system, and a flow cell containing a plurality of fluorescent nitrogen-vacancy nanodiamonds immobilized on a surface of the flow cell. Each of the plurality of fluorescent nitrogen-vacancy nanodiamonds is conjugated to a probe that is also bound to a magnetic nanoparticle. The probe specifically binds to a target material when present in a biological sample and a conformational change in the probe results in a change in the distance between the probe-bound fluorescent nitrogen-vacancy nanodiamonds and the probe-bound magnetic nanoparticle that is detectible evidencing the presence of the target material in the sample.

### Systems, Compositions and Reagents

Provided are systems, compositions, reagents, processes, and devices for detection of target materials in biological samples employing diamond magnetometry.

Embodiments of the present disclosure, for example, in comparison to concepts failing to include one or more of the features disclosed herein, provide decreased detection time requirements, lower cost, equivalent or lower limits of detection, or combinations thereof.

n some exemplary embodiments, a system for detection of a target material includes a plurality of fluorescent nitrogen-vacancy nanodiamond-probe-reporter conjugates (MND-probe conjugates). Each MND-probe conjugate includes at least one fluorescent nitrogen-vacancy nanodiamond (FND). Each MND-probe conjugate also includes at least one reporter. Each MND-probe conjugate also includes at least one polymeric probe including a biomolecule. The polymeric probe has binding specificity to the target material. Each of the at least one FND and the at least one reporter is linked to the at least one polymeric probe. The reporter is removed from proximity to the FND to result in a detectable change. In other words, a distance between the FND and the reporter in the MND-probe conjugate changes when the at least one polymeric probe binds to the target material and displaces or releases the target material.

Diamond magnetometry (DM) is a quantum-based sensing technology capable of screening biomarkers of breast cancer, potentially using a liquid biopsy. DM permits the sensitive detection of synthetic microRNA oligonucleotide targets (miR-21, miR-421 and miR-486 and miR-1303). DM is faster than quantitative reverse transcriptase polymerase chain reaction (RT-qPCR), does not require nucleic acid amplification, uses no expensive components, and can currently achieve sensitivities in the range of 10⁻¹² to 10⁻¹⁴ M for microRNA targets. The technology can be automated, miniaturized, and validated for high throughput screening using a microarray format.

More specifically, DM is an all-optical procedure that uses a magnetometer, fluorescent nitrogen-vacancy center nanodiamonds (FNDs) and magnetic nanoparticles (MNPs) to detect the magnetic field generated by MNP. DM allows quantitative detection of many different parameters including electron and nuclear spins. DM has been used to detect free radicals, chemical reaction products, virions as well as sense local environmental parameters such as pH, magnetic noise, temperature, and 3D rotations of specific molecules. Single molecule detection of biotin-avidin complexes has also been demonstrated with attomolar sensitivity. Surprisingly, and despite certain biological demonstrations, application of DM for cancer screening has not been reported.

The nitrogen vacancy (NV) center of diamond is a crystallographic defect within the diamond lattice where a nitrogen atom replaces a carbon atom and is adjacent to a vacancy site. This structure emits photostable fluorescence, in the NIR region, when excited with green light. NV-centers are unique in that the electron spins associated with fluorescence can be polarized and are magnetically sensitive. Polarization changes can be detected by sweeping a microwave field (from 2.7 to 3.0 GHz) across the NV-center while measuring fluorescence. If a magnetic or paramagnetic field is close by, it alters the polarized state of the NV-center which changes the fluorescence. Thus, NV-centers offer unparalleled sensitivity in detecting small magnetic fields.

Förster Resonance Energy Transfer (FRET) is a non-radiative energy transfer event that involves dipole-dipole coupling between an excited state donor fluorophore and a ground state acceptor, which can be either another fluorophore or a quencher. FRET requires both spectral overlap and proximity between the donor and acceptor. Thus, the donor and acceptor molecules for FRET needs to be carefully matched. FRET spectroscopy is useful for nanoscale measurements provided that the distance between the donor and acceptor pairs is not more than 10 nm. FRET exhibits strict distance dependence which drops rapidly as the inverse-sixth-power of the distance.

In contrast, DM is based on spin-spin coupling of a paramagnetic nanoparticle rather than dipole-dipole coupling between two organic molecules, which is typical in FRET. The nanoparticle can be of any size but should have a free pair of electrons. However, in some instances, ions can be used to affect an NV-center fluorescence. Furthermore, diamond magnetometry, as performed with nitrogen-vacancy (NV) centers in diamond, have been used for detecting magnetic fields across a range of length scales including a few nanometer to hundreds of nanometer scale.

The equipment needed for FRET is considerably different from that of DM. A basic FRET experiment can be performed in commercial equipment such as filter-based or grating-based fluorescence plate readers for conventional fluorescence intensity measurements. For lifetime measurements, a commercial cuvette or fluorescence lifetime imaging microscopy (FLIM) is used. These instruments cannot be used for DM.

For DM, a custom-built magnetometer is typically used. This basic instrument includes a fluorescence spectrophotometer, a microwave generator and in some cases a Helmholtz coil is used. A laser is used to provide high density power and an acousto-optic modulator (AOM) is included for pulsed-laser sequences.

The development of DM for biomedical applications would have an immense impact in medicine and provide a new tool for researchers looking for a rapid and inexpensive alternative to reverse transcriptase-polymerase chain reaction (RT-PCR) approaches. More specifically, the successful demonstration of this technology in medical screenings for breast cancer would usher in the use of DM for monitoring other types of cancers, as well as biomarkers associated with health and wellbeing. The development of DM for biomedical applications would have an immense impact in medicine and transform medical diagnostics in revolutionary and unprecedented ways.

Breast cancer significantly impacts women's and men's health in the United States and around the globe. The American Cancer Society estimates rates of new cases of breast cancer at approximately 120 cases per 100,000 women and, during 2022, 287,850 new cases of invasive breast cancer were reported in the US. Early detection of breast cancer improves the likelihood of successful cancer therapy, offers broader treatment options and a better overall quality of life for patients. Mammography remains the only standard of care for diagnostic breast cancer screening, but its reduced sensitivity as mammographic breast density increases, potential health risks of ionizing radiation, and patient discomfort during mammography are important drawbacks. This highlights the need for additional, minimally invasive tests for early detection of breast cancer, treatment monitoring, and post-treatment follow-up.

The classification and therapy of breast cancer is generally based on tumor staging/grading and the presence/absence of protein biomarkers, such as estrogen receptor (ER), progesterone receptor (PR), epidermal growth factor receptor 2 (HER2) and Ki-67 (proliferation marker). Based on these expression patterns, breast cancer is classified into four main subtypes: Luminal A (ER⁺, PR⁺, HER2⁻, Ki-67 low), Luminal B (ER⁺, PR^{+/-}, HER2^{+/-}, Ki-67 high), HER2-enriched (ER⁻, PR⁻, HER2+) and triple negative (TNBC/ER⁻, PR⁻, HER2⁻). Subtypes differ in their prognosis and outcome, which illustrates the importance of using biomarkers to understand and manage breast cancer.

MicroRNAs (miRNAs) are a large class of non-coding RNAs, typically approximately 22 nucleotides in length, which are involved in the post-transcriptional silencing of messenger RNAs (mRNAs). MiRNAs bind to the mRNA 3-prime untranslated region, which results in mRNA cleavage, degradation, or translational repression. Given their critical role in regulating gene expression it is not surprising that aberrant expression of miRNAs is associated with the onset and progression of breast cancer. MiRNAs can have different roles in cancer, and these can be described as oncogenic or tumor-suppressive. Upregulation of oncogenic microRNAs can inhibit expression of tumor-suppressive genes, while downregulation of tumor-suppressive microRNAs can increase signaling pathways that enhance tumor development.

MiRNAs have properties that make them good diagnostic biomarkers for liquid biopsy (LB), as they remain stable even under severe conditions such as pH variations or multiple freeze-thaw cycles. Additionally, they are found in different body fluids, either free, bound to proteins, or encapsulated in exosomes, and this facilitates their extraction and analysis. However, miRNAs are a challenging target to detect by conventional RT-qPCR, due to their small size, low abundance, and sequence homology among family members. There are also methodological challenges and quantification issues to overcome before miRNAs become clinically valid biomarkers. Despite these concerns there remains considerable interest in the use of miRNAs as an analytical tool in clinical practice.

Liquid biopsy (LB) refers to diagnostic tests that are performed on biological fluids, such as serum or plasma. LB has gained significant attention in cancer diagnosis as it is a non-invasive medical test that can identify biomarkers released by tumors into the bloodstream. Compared with solid biopsy, LB allows more comprehensive diagnostic testing and holds significant promise for early detection of disease, monitoring treatment response, and identifying potential therapeutic targets. Importantly, a number of microRNAs are deregulated in the plasma of breast cancer patients when compared to healthy individuals, including miR-10b, miR-21, miR-145, miR-155, miR-19a, miR-21, miR-24, miR-155, and miR-181b, miR-195, miR-16, and let-7a.

According to embodiments of the invention, methods and systems are disclosed which would permit the use of DM to rapidly analyze multiple miRNA targets in an array format, for example, to test liquid biopsies.

A magnetometer device senses a magnetic field, measuring its strength and direction. A diamond magnetometry device can measure T1, T2, and ODMR of fluorescent nitrogen-vacancy (NV) diamonds, and measure nanoscale magnetic fields, such as from magnetic particles, magnetic nanoparticles, or other magnetic materials close to the NV diamonds.

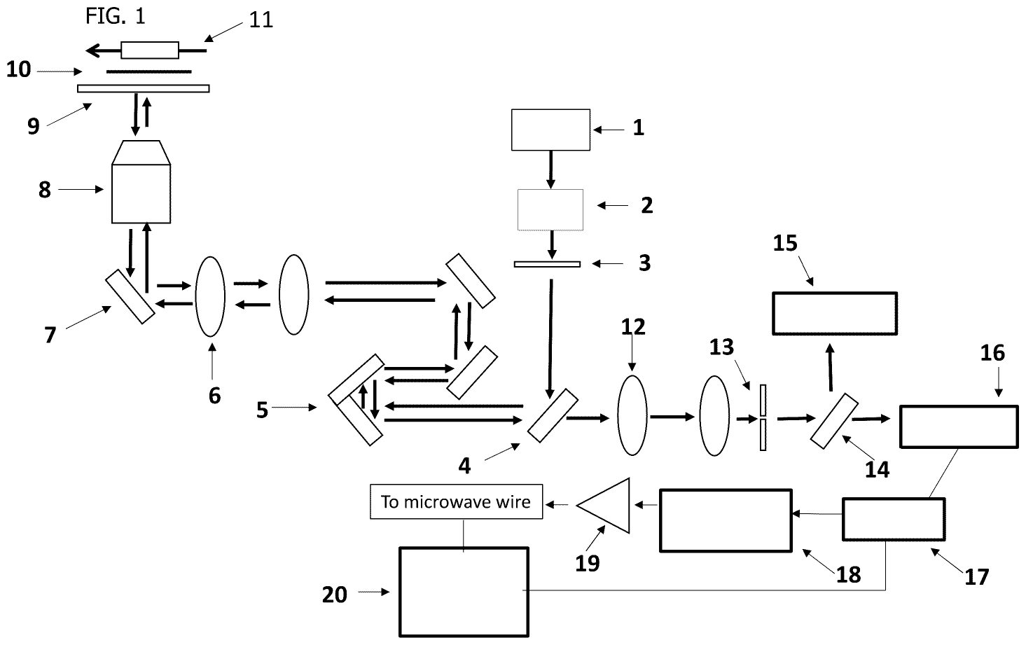

FIG. 1 schematically shows optical components for ODMR and T1 measurements in a diamond magnetometry device. Green (532 nm) laser light 1 passes through an acousto-optic modulator (AOM) 2 and then through an iris 3, a dichroic mirror 4, a galvo scanner 5, and various lenses 6 and mirrors 7 before being focused on an objective 8, such as, for example, a 100× objective. To achieve a Gaussian laser beam, a spatial filter is placed at the beginning of the beam path. In short, the laser coming directly from the head is focused to a point with a 20 -cm lens, and a 100 -μm pinhole is located at the focus point followed by the iris 3. The iris 3 is closed down to block all but the central diffracted spot of the beam, which is then collimated by a second 20 -cm lens. This results in a much more uniform beam going to the objective and a more stable reading from the sample.

The beam excites NV nanodiamonds that are affixed to a glass coverslip bonded to the top of a flow cell 11 that is mounted on a microscope stage. The flow cell 11 is positioned close to a thin microwave (MW) wire. The resulting emission fluorescence passes through the same set of mirrors and lenses and the emission fluorescence is filtered from the excitation laser by a 560-nm longpass (LP) filter 12 and passes through a 75-μm pinhole 13 to isolate the focal plane. The fluorescence is then split by a beam splitter 14 and sent either to a charge-coupled device (CCD) camera 15 for spectral analysis or to a photon counter 16. The image is processed by a computer 17 to produce the ODMR spectrum at a selectable region of interest. The computer also controls the MW source 18. The MW power amplifier 19 supplies about 1 Watt through a thin wire placed near the diamonds 10 and is analyzed by a MW analyzer 20.

The optical system for detecting and monitoring changes in NV-center T1, T2, and ODMR is built on an optical table with vibration isolation supports. It consists of a few major components which can be re-positioned and replaced with other components depending on whether T1, T2, or ODMR are being measured. A data acquisition card is used to record the experimental data. T1 and T2 measurements do not require the microwave components.

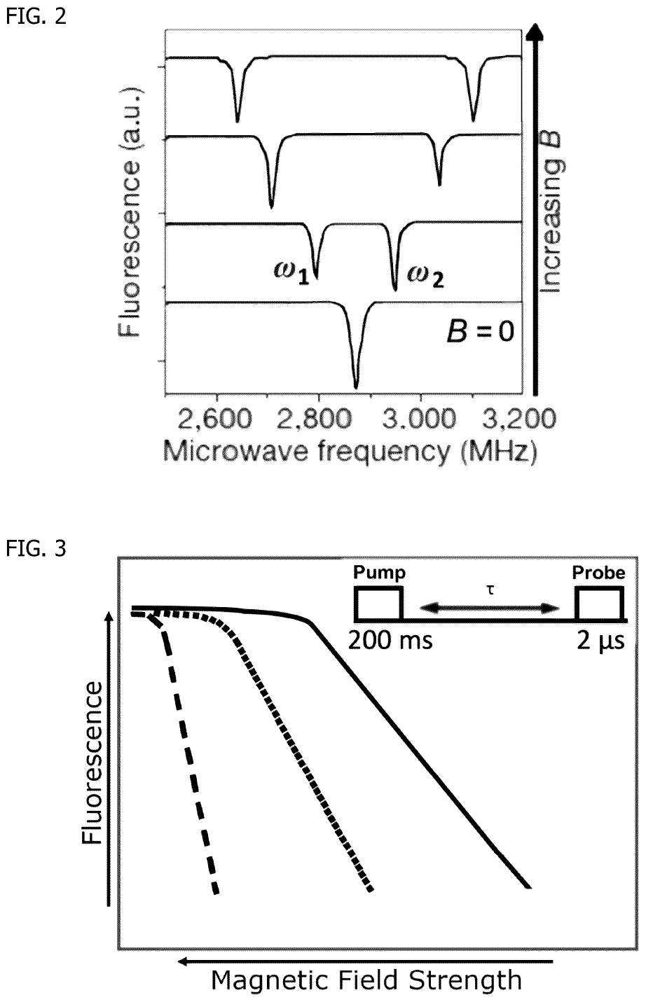

Referring to FIG. 2, the flow cell 11 includes a chamber body 21 with a sample inlet 22 leading to a hollow test chamber 23. A ridge 24 in the test chamber 23 formed into the chamber body 21 forces a sample fluid into close proximity with the cover slip 25 on the center of which an array of microdiamonds 26 is affixed. A depression 27 surrounding the test chamber 23 contains a gasket, which confines the sample fluid to the test chamber 23. A heating pad 28 applies heat, as needed, to increase reaction rates. The sample fluid is injected, such as, for example, by a sample syringe pump 29, into a mixing chamber 30, where it mixes with buffer also injected, such as, for example, by a buffer syringe pump 31, into the mixing chamber before flowing through the sample inlet 22 and into the test chamber 23. The buffer may be any appropriate pH buffer solution, such as, for example, phosphate-buffered saline (PBS) of a pH of about 7.5.

In some embodiments, a fluorescent nitrogen-vacancy nanodiamond (FND) has a size in the range of about 20 nm to about 100 nm, alternatively in the range of about 20 nm to about 80 nm, alternatively in the range of about 30 nm to about 60 nm, alternatively in the range of about 40 nm to about 50 nm, or any value, range, or sub-range therebetween.

In some embodiments, the FND has 1 to about 100 nitrogen-vacancy centers planted at a depth of about 5 to about 15 nm below the surface of the FND. The nitrogen-vacancy (NV) color center of fluorescent diamond, an impurity of a nitrogen atom adjacent to a vacant lattice site, is paramagnetic, making it responsive to other magnetic species. In exemplary embodiments, the surface of the FND is free or substantially free of scavengers of diamond fluorescence. In exemplary embodiments, the surface of the FND is modified to include functional groups that permit coupling of probes to the FNDs, including nucleic acids or amino acids or combinations thereof to the surface. In some embodiments, the surface chemistry includes amines, triple bonding moieties such as for example propargyl group, or azides for click chemistry.

Excitation of an NV-center with green light results in a broadband photoluminescence emission with a zero phonon line (ZPL) at 575 nm and 637 nm with longer wavelength emissions extending into the infrared region. Diamonds containing NV-centers exhibit very long spin coherence times, making them extremely sensitive to magnetic fields, and this can be followed optically via ODMR and T1 and T2. Diamond magnetometry encompasses the T1, T2, and ODMR of NV diamond and can detect minute changes in magnetic field.

The NV center has two charge states: neutral, NV⁰ and negative, NV⁻, of which the negative charge state is relevant for magnetometry. The negative charge state (NV⁻) forms a spin triplet ground state having three spin sublevels (mₛ=−1, 0, or +1). Excitation with green light optically polarizes the NV center into the mₛ=0 sublevel (bright state) which scatters about 30% more photons than the mₛ=±1 states. When a resonant microwave field induces magnetic dipole transitions between these electronic spin sublevels, it disrupts the optically pumped spin polarization, resulting in a significant decrease of the nitrogen-vacancy center fluorescence, as shown in FIG. 3 at B₀), which is the ODMR. Due to symmetry, the mₛ=±1 sublevels of the nitrogen-vacancy defect are degenerate at zero magnetic field (B=0), resulting in a single resonance line appearing in the ODMR spectrum at B₀ and at 2,870 MHz, as shown in FIG. 3. If an external magnetic field is encountered, from a magnetic particle for instance, it lifts the degeneracy of mₛ=±1, leading to the appearance of two lines from which the external magnetic field can be measured by the distance between the two lines. As the magnetic field decreases, by increasing distance between the diamond and the MP for instance, the separation between the ODMR lines decreases. The splitting of the ODMR spectrum into separate components by a static magnetic field is called Zeeman splitting. In general, micron-sized NV-center diamonds can detect magnetic fields below 1 nT.

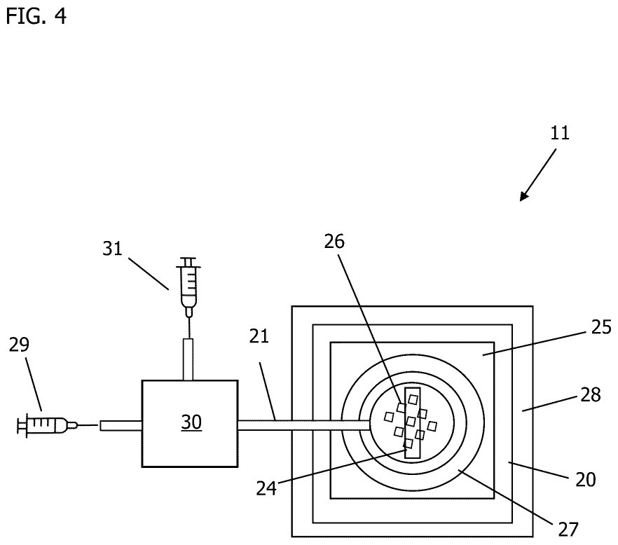

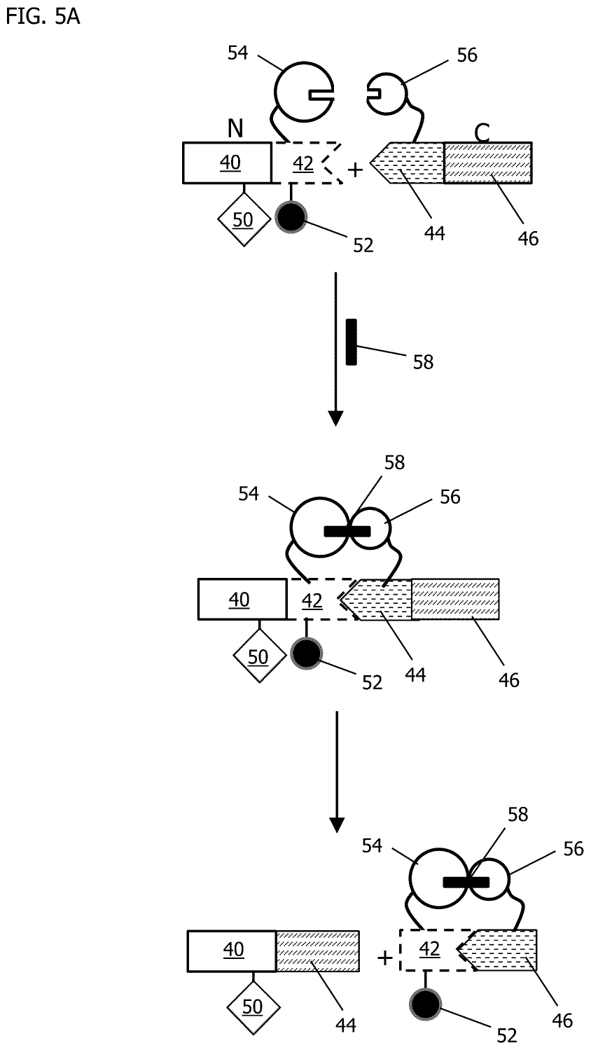

T1 and T2 of NV-centers are also sensitive to fluctuating magnetic fields, as shown in FIG. 4. T1 represents the decay lifetime for a population of excited NV-centers to return to the ground-state, and T2 represents the cumulative loss of at least 63% of original phase coherence. Unlike ODMR, T1 and T2 relaxation can be measured without the need for microwave excitation, which makes relaxometry faster to perform.

Briefly, a T1 relaxometry study involves applying a pulse probe sequence, as shown in FIG. 4, in which the NV-center diamond is given a millisecond laser pulse (532 nm) to preferentially populate the mₛ=0 sublevel, as described above. The laser is then shut off, and during this dark time (τ), the system relaxes back towards the equilibrium condition. After a specified dark time, the diamonds are given a short, microsecond laser probe and the population remaining in the mₛ=0 sublevel is read out by monitoring the intensity of the emitted red light.

A T2 relaxation study is performed similarly, but the cumulative loss of fluorescence is monitored, rather than recovery of the equilibrium condition. FIG. 3 shows a typical relaxation curve for an NV-center diamond in the absence of a magnetic field. In the presence of strong magnetic noise, such as paramagnetic gadolinium ion (Gd⁺³), relaxation time becomes much shorter. This is because the magnetic noise from Gd⁺³ interferes with the optically pumped spin polarization, which resets the equilibrium condition of T1 and the net loss of spin coherence of T2.

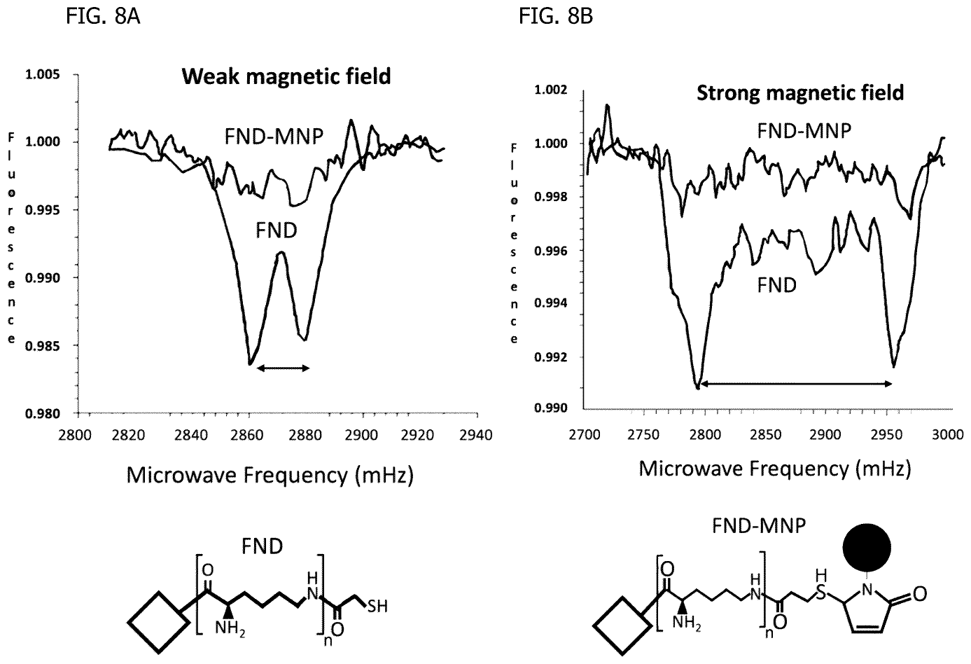

In exemplary embodiments, diamond magnetometry detects specific materials in biological samples. In exemplary embodiments, the distance between NV diamonds and reporters is controlled, and the response of the ODMR, T1, and T2 NV diamonds is measured as the distance is changed. Examples 1 and 2 and FIG. 8A and FIG. 8B show how distance between and NV diamond and reporters, where the reporters are magnetic particles, affects ODMR, T1, and T2.

In exemplary embodiments, the reporters are magnetic nanoparticles having a size in the range of about 10 nm to about 100 nm, alternatively in the range of about 10 nm to about 80 nm, alternatively in the range of about 20 nm to about 60 nm, alternatively in the range of about 30 nm to about 50 nm, or any value, range, or sub-range therebetween.

Appropriate materials for the magnetic nanoparticles may include, but are not limited to, iron oxide, maghemite, magnetite, a diamagnetic material, a supermagnetic material, a ferromagnetic material, a ferrimagnetic material, a quantum dot, an upconverting material, ferritin, a ferritin-like protein, a heme-containing protein, an oligonucleotide-containing magnetic material, and a core-shell including a magnetic core and an outer shell of a component such as silane, polysaccharide, gold, polymer, or dendrimer.

In exemplary embodiments, a diamond magnetometry system measures magnetic properties of fluorescent NV nanodiamonds conjugated with a probe for a targeted material that is exposed to a biological sample to determine the presence, identity, and/or amount of the target material in the sample.

The NV center of the diamond includes a point defect in the diamond crystal lattice that emits photostable red fluorescence following excitation with green light. The ODMR of an NV diamond, which is the decrease of fluorescence in the presence of a resonant microwave field, exhibits measurable variation based upon the proximity and strength of a magnetic field. The T1 and T2 of NV diamonds are also measurably responsive to the presence of, strength of, and proximity to, magnetic fields.

In exemplary embodiments, a composition includes NV microdiamonds with tethered probes that can control the distance of MPs from their surfaces.

In some embodiments, the presence of a target material alters the conformation of the probe, changing the distance between the MP and NV diamond, such as, for example, moving them further from each other.

In some embodiments, the presence of a target material activates the probe's attachment to, or release from, other materials that are connected to the MP, changing the distance between the MP and NV diamond, such as, for example, moving them either closer to or further from each other.

In some embodiments, the changes in T1, T2, and ODMR of the NV diamond that results from the change in distance from the MP indicate the presence of a target of interest in a sample.

To control the distance between the NV diamonds and MP, a probe is attached to the NV diamond surface that can capture and hold the MP near the NV diamond surface, move the MP a predetermined distance from the surface, or release the MP from the NV diamond completely. In some embodiments, the probe includes a nucleic acid or an amino acid. In some embodiments, the nucleic acid includes a stem-loop structure. In some embodiments, the nucleic acid includes an aptamer. In some embodiments, the amino acid is part of an intein-extein system. In some embodiments, the amino acid is part of an antibody.

In some embodiments, the probe includes a nucleic acid conjugated to the fluorescent nanodiamonds. In some embodiments, the probe nucleic acid is a single stranded oligonucleotide up to 50 bases in length. In other embodiments, larger DNA fragments may be used. In some embodiments, the conjugation is accomplished with amines. In some embodiments, the conjugation is accomplished with azides. In other embodiments, other functional groups may be used to accomplish the conjugation, depending on the availability of functional groups from nucleic acid manufacturers. In some embodiments, the conjugation is at the 5′ and/or the 3′ end of the oligonucleotide. In other embodiments, the conjugation may be through a functional group on an internal base of an oligonucleotide. In other embodiments, other functional groups and chemistries may be used. The oligonucleotides may be DNA oligonucleotides, RNA oligonucleotides, or other oligonucleotides of DNA and/or RNA containing modified bases. Appropriate modified bases may include, but are not limited to, 2′-o-methoxyethyl bases (2′-MOE), which are used for antisense oligos (ASO), aptamers, and small interfering RNA (siRNA); 2′-o-methyl RNA bases; 2-aminopurine; 5-bromo deoxyuridine; deoxyuridine; 2,6-diaminopurine; dideoxycytidine; deoxyinosine; 5-methyl deoxycytidine; 5-nitroindole; 5-hydroxybutynl-2′-deoxyuridine; or 8-aza-7-deazaguanosine.

In one embodiment, the method for controlling distance includes linking fluorescent NV-center nanodiamonds (NV diamonds) to reporters, such as, for example, magnetic particles, magnetic nanoparticles, or other magnetic materials (MPs), through single-stranded DNA having a stem-loop structure, as shown schematically in FIG. 11 and described in more detail in Example 4. The stem-loop includes a single-stranded loop region that is flanked by self-complementary termini which hybridize to form the stem. One terminus of the stem region is attached to the NV diamond, and the other end is attached to the MP. The stem region of the stem-loop structure positions the MP close to the NV diamond, and thereby disrupts polarization of the NV-center. This produces Zeeman shifts which are observed as at least two dips in the ODMR spectra.

The DNA in the loop region is complementary to, and will hybridize with, the target DNA being detected. Hybridization of target polynucleotide to the loop region DNA causes the loop to open, thereby linearizing the stem-loop structure and moving the MP away from the nanodiamond surface. Since the distance (r) over which the magnetic field of the MP influences the NV-center is extremely short (1/r³), and the DNA sequence is long, the system returns to a zero magnetic field state and the resulting microwave sweep should produce only the zero-field resonance dip at 2,870 kHz in the presence of the target material.

In some embodiments, a stem-loop design of a nucleic acid probe is used in detecting nucleic acids target materials in a sample, as shown schematically in FIG. 11. As depicted, the stem-loop is a single-stranded DNA containing complementary DNA sequences at the 5′ and 3′ ends. The ends form a short double strand structure with the single-stranded loop region positioned between the two complementary sequences. Hybridization of a DNA/RNA sequence in the loop region destabilizes the stem structure and thus linearizes the DNA.

In some embodiments, magnetic nanoparticles are used to affect the Zeeman splitting separating the mₛ=+1 from the mₛ=−1 states. However, other approaches can also be used to affect the excited NV-center, including the use of nitroxide spin labels, and ions such as Gd⁺³, or Mn⁺², and the protein ferritin. In the case of Gd⁺³, the ion is chelated within the macrocycle 2,2′,2″,2″′-(1,4,7,10-tetraazacyclododecane-1,4,7,10-tetrayl)tetraacetic acid (DOTA). This macrocycle, in turn is coupled to the FND through a covalent linkage. For example, an N-hydroxysuccinimide (NHS)-DOTA can be purchased from a commercial supplier (Macrocyclics, Pano, TX) and then linked to a pegylated-FND having an amine at the terminal end of the PEG molecule.

In another embodiment, the same flow cell may be used, but the ODMR analysis is done in an optical system that creates a biased magnetic field. The presence of the magnetic field produces a Zeeman shift in both the unhybridized and hybridized states of the DNA probe thereby affecting the distance between the FND and the MP. In this method, the close approach of the magnetic nanoparticle further broadens the Zeeman splitting, by an amount equal to the size of the magnetic nanoparticle field strength.

The field strength of the MP affects Zeeman splitting and this is influenced by its composition. The MP may be composed of a metal core including FeO, Fe₂O₃, Fe₃O₄, Co, Fe, Ni, CoFe, NiFe, CoO, NiO, or ferrites including MFe₂O₃, where M includes either Co or Ni. For biological applications MPs are coated with biocompatible molecules or polymers such that functional groups are available for conjugation. Under most conditions a commercial MP can be used, consisting of Fe₃04 and having carboxyl groups on the surface. Another magnetic entity that could also be used is the gadolinium+3 ion.

One method of creating a stem-loop DNA for quantum-based sensing is illustrated in FIG. 11. The alcohol groups on glycidol-coated nanodiamonds are converted into NHS esters using DSC. These are then reacted with an azido-PEG11-amine (Broadpharm, San Diego, CA). The result of this procedure is the creation of a FND with azide groups on the surface. This group can be confirmed by FTIR. A stem-loop DNA sequence may be designed and tested for stable structures using the manufacturer's software (Integrated DNA Technologies). The single-strand DNA may be designed with a propargyl group at the 5′ ends and an amine group at the 3′ end. The DNA is validated by mass spectrometry. The surface functionalized FNDs are contacted with the 5′ propargyl modified DNA strand to accomplish conjugation and form the DNA-FND conjugate.

In some embodiments, the nucleic acid of the probe is part of an aptamer. DNA aptamers are short single-stranded stretches of nucleic acids that form unique hairpin-like DNA structures that selectively bind specific protein target materials. Thus, they behave as antibodies. A feature of aptamers that can be exploited for ODMR is that they undergo conformational changes upon binding a target protein. The binding event translates into changes in spatial distance between the NV-center diamond and a magnetic nanoparticle, which affects ODMR spectra.

For example, cortisol is a glucocorticoid hormone produced in the adrenal gland and secreted as part of the flight or fight impulse in response to fear or stress but has other roles in mental health (stress and depression) and emotional events. In situations of chronic stress and disrupted sleep-wake cycles, the adrenal glands secrete an abnormal amount of cortisol in an abnormal rhythm. Monitoring cortisol levels is a useful measure of normal or abnormal conditions.

The DNA sequence (38-mer) for a possible cortisol aptamer, for example in the aptamer-“A-probe” complex depicted in FIG. 17, which aptamer sequence is 5′-GCCCGCATGTTCCATGGATAGTCTTGACTAGTCGTCCC-3′ (SEQ ID NO: 1) and has a limit of detection of less than 1 ng/mL. Importantly, this aptamer sequence undergoes a significant conformational change upon binding cortisol, which makes it useful for ODMR. This aptamer is available as an oligonucleotide with an azide group on the 5′ end and a terminal amine group on the 3′ end. The same stem-loop approach described above may be used here, meaning that binding of the ligand, (cortisol) causes a conformational change in the DNA, which produces positional changes in the MP-FND spatial relationship in the MND-probe conjugate. As used herein, the term “A-probe” refers to the complementary oligonucleotide that binds to the aptamer and carries one of the FND and the MP wherein the aptamer carries the other of the FND and the MP to provide the sensor. The sequence for the A-probe as depicted in FIG. 17 may be 5′-ATACCGAGAGCCCTGCTG-3′ (SEQ ID NO: 7) or having such other sequence that is complementary to the aptamer and binding affinity for the target, in this case, cortisol.

In some embodiments, a nanodiamond is spotted on the surface of a glass coverslip, which is then mounted on a flow cell. The flow cell is positioned over the microscope objective and the nanodiamonds are excited with a green laser. ODMR spectra are recorded as fluid is pumped across the flow cell. A test sample is then passed across the flow cell, where it interacts with the nanodiamond. A positive detection, representing capture of the cortisol molecule, is either a change in the magnitude of the Zeeman shift in a biased magnetic field or a change in the contrast of the resonance peaks in a non-biased magnetic field. The cortisol may be then stripped from the aptamer-FND conjugate using a series of laser pulses to heat the DNA such that it denatures and releases the cortisol molecule. The aptamer may be renatured to its original conformation by slowly cooling the aptamer conjugate.

In other embodiments, the aptamer is a split aptamer. In such embodiments, one of the ND and the MP may be attached to one half of the split aptamer with the other of the ND and the MP being attached to the other half of the split aptamer. Hybridization of the split aptamer indicating the presence of a target material brings the ND and MP close together as determined by T1 and T2 and/or ODMR measurements.

In some embodiments, the probe includes an amino acid conjugated to the fluorescent nanodiamonds. In some embodiments, the amino acid includes an intein or an extein. In some embodiments, the amino acid includes an antibody or an active portion of an antibody.

In some embodiments, the method of controlling distance between NV diamond and MP is their attachment to inteins, with their proximity being controlled using protein cis- or trans-splicing. An intein is an internal protein segment removed from between two exteins, an N-extein and a C-extein, during protein splicing.

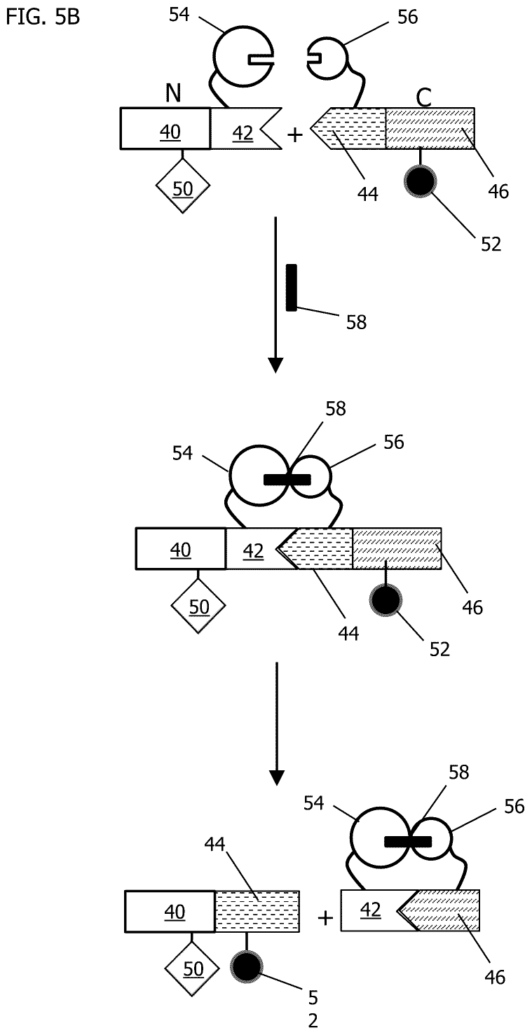

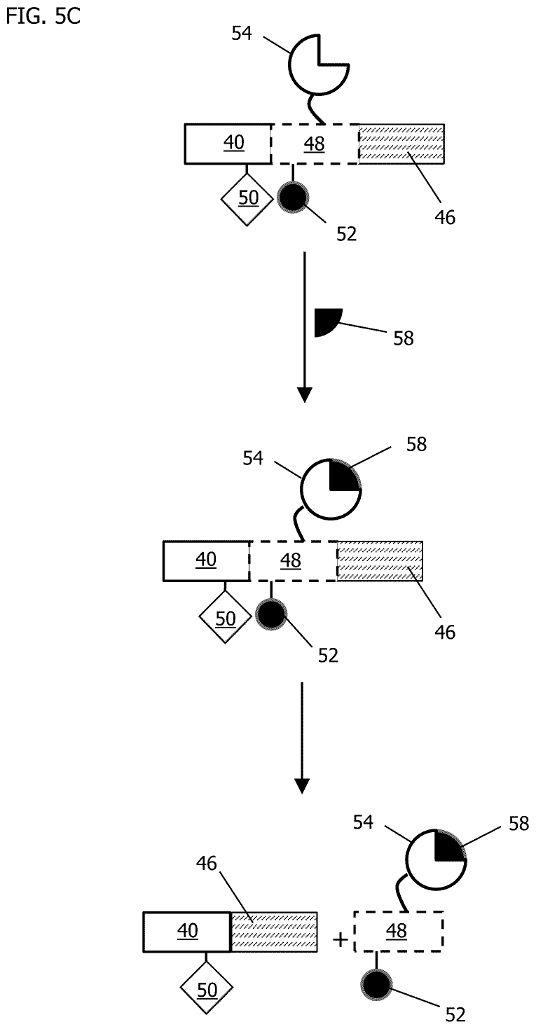

In protein trans splicing (PTS), an intein has been split into two fragments and reassociation of the fragments is required prior to splicing of the protein. Trans-splicing inteins have been engineered to undergo conditional protein splicing, or CPS. CPS requires the addition of a trigger to initiate splicing of a precursor fusion protein. For CPS of trans-splicing inteins, reassociation can be conditional on presence of a small molecule, such as a target material. FIG. 5A, FIG. 5B, and FIG. 5C show some options for intein-extein probes.

Referring to FIG. 5A, an MP 52 is held near an NV diamond 50 surface by attaching either an NV diamond or MP to the intein 42, 44 and the other to the extein 40, 46. When the target material 58 is encountered, it is bound by the receptors 54, 56, and the intein 42, 44 is excised from the extein 40, 46, and the MP 52 is removed from the NV diamond 50 surface. The resulting changes in the NV diamond's T1, T2, and ODMR measurements indicate the presence of the target material 58 in a sample.

Referring to FIG. 5B, an NV diamond 50 is attached to one extein 40 bound to an intein fragment 42 and the MP 52 to the other extein 46 in another configuration using PTS. Upon binding of the target material 58 by the receptors 54, 56, and subsequent reassociation of intein fragments 42, 44, resulting in excision of the intein 40,46, the MP 52 is held near the NV diamond 50 surface, again affecting the T1, T2, and ODMR of the NV diamond 50.

Referring to FIG. 5C, detection via cis-splicing is similar to that of trans-splicing, however the intein 48 is intact instead of split. In this case, the target 58 binding to the receptor 54 causes excision of the MP-bound intein 48 that is positioned between two exteins 40, 46, again removing the MP 52 from the NV diamond 50 surface.

In some embodiments, the probe includes an antibody. In such embodiments, the method of controlling distance between NV diamond and MP is by their attachment to antibodies and the selective binding between antibodies and their antigens.

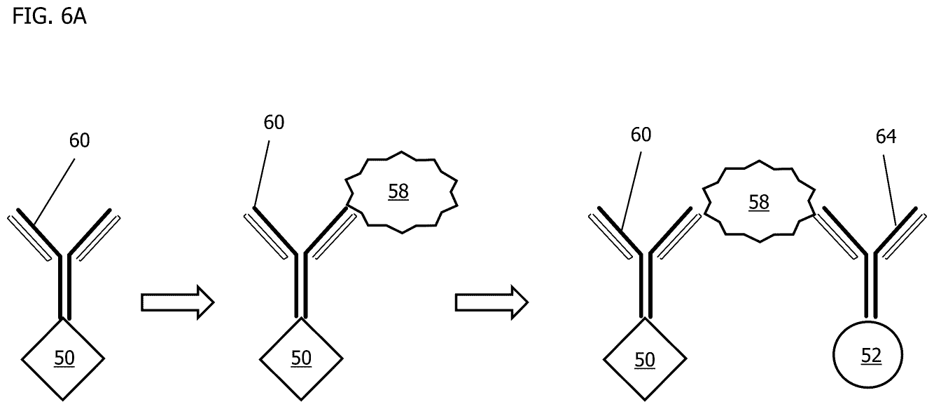

Referring to FIG. 6A, the probe on the plated-coated NV diamond 50 includes an antibody 60. The coated plate is then exposed to a biological sample. If the target material 58, antigen in this case, is present, it binds to the antibody 60. Antibody-bound MP is then added to the system and binding between the antibody 64 on the MP 52 and the antigen 58 bound to the antibody 60 on the NV diamond 50 brings the NV diamond 50 and MP 52 into close proximity for detection by T1, T2, and/or ODMR.

Referring to FIG. 6B, a plate is coated with a capture antibody 70. The coated plate is then exposed to a biological sample. If the target material 58, antigen in this case, is present, it binds to the capture antibody 70. NV diamond 50 with a detecting antibody 72 probe is then added, and the detecting antibody 72 binds to the antigen 58, if present. Finally, MP 52 with a bound secondary antibody 74 is added, and the secondary antibody 74 binds to the detecting antibody 72, if antigen 58 is present, to bring NV diamond 50 and MP 52 into close proximity for detection by T1, T2, and/or ODMR.

Detection of a target material in a biological sample by the compositions, devices, and systems disclosed herein may have many different applications. In some embodiments, the compositions, devices, and systems described herein are used for monitoring in a biomedical application. Such applications may include, but are not limited to, the detection and measurement of health-related biological indicators in body fluids, such as, for example, for cancer diagnosis or cardiovascular applications, monitoring for presence of environmental pathogens and chemicals, or monitoring of medical treatment efficacy or decontamination effectiveness.

Such monitoring of human performance may include detection of stress indicators, such as, for example, cortisol, lactate, or urea in sweat. Monitoring may additionally or alternatively include detection of other health indicators, such as, for example, protein biomarkers, DNA, RNA, or microRNA that would be evidence of health concerns, such as, for example, pathogen exposure, stress, adverse cardiac events, or diseases such as cancer or diabetes. Some embodiments include single molecule detection to identify indicators that are present in minute quantities, making early detection and treatment possible. Indicators may be identified in body fluids, such as, for example, blood, sweat, saliva, or urine.

In some embodiments, monitoring of medical treatment efficacy includes measuring indicator levels following treatment, which permits the development of new treatments by tracking indicator levels following sample treatment with various potential therapies. By use of an array of therapy-activated fluorescent diamonds, multiple therapies may be analyzed for efficacy simultaneously.

In some embodiments, the detection is of an miRNA target related to a human breast cancer cell line in a liquid biopsy. DM shows specificity in distinguishing between the miRNA oligonucleotide miR-21 and miR-421, miR-486, and miR-1303. Moving from a well-defined system to total RNA prepared from human serum and plasma and total RNA isolated from breast cancer cell lines (MCF7, MDA-MB-231 and SK-BR-3), their expression levels may be quantified. Both miRNAs that have been tested and other miRNAs that are implicated with breast cancer, including, but not limited to, Let-7a, miR-195, and miR-145, may be detected. In addition to the use of DM for analyzing and quantifying miRNA, the results may be compared against RT-PCR studies of the same RNA preparations.

In some embodiments, DM detects the presence of approximately 1 ng miRNA against a background of 10,000 ng of total RNA prepared from cancer cell lines and in liquid biopsy samples with a p-value<0.05, and with 95% confidence limits.

In some embodiments, quantification of miRNA by DM approximates RT-PCR results with at least 95% of the sensors increasing their ODMR contrast upon hybridizing to their RNA target.

In some embodiments, DM detects miRNA targets ten times faster than RT-qPCR with a false-positive detection rate of less than 2%.

Multiplexing can greatly simplify measuring multiple samples of the same type. In some embodiments, a custom-built liquid handling system prints arrays in a 5×5 format on glass coverslips and binds the sensors directly on the coverslip surface. In some embodiments, fluorescence is used to characterize the arrays for uniformity and stability and validate the arrays using purified miRNAs for specificity, sensitivity, and any cross contamination between spots. Finally, the array format may be used to determine miRNA expression patterns in blood from a limited number of human breast cancers samples.

In some embodiments, there is less than 5% variability between spots for sensors printed across 5×5 arrays as determined by fluorescence intensity and ODMR contrast results.

In some embodiments, such microarrays detect as little as 1 ng miRNA against a background of 10,000 ng total RNA prepared from LB with the decreased ODMR processing times of less than 15 minutes for a 5×5 array containing 25 samples.

In some embodiments, a liquid biopsy demonstrates statistically significant differences in miRNA expression levels for at least one miRNA target between normal and breast cancer patients.

In some embodiments, single strand DNA is coupled to a reporter, such as, for example, a paramagnetic nanoparticle (MNP), and a complementary strand to an FND. Hybridization between two complementary DNA strands brings the MNP close to the FND. The magnetic field varies as 1/r⁶ with paramagnetic nanoparticle separation meaning that picometer changes in magnetic position can be sensed instantaneously through a change in ODMR contrast at the resonance frequency around 2.86 GHz. Such positional changes between the FND and MNP are achieved by a toehold RNA strand displacement strategy.

In some embodiments, a coating process creates both amines and propargyl groups on the same nanodiamond, which allows the coupling of both DNA oligonucleotides and the biotin binding molecule, streptavidin, to the same FND.

In some embodiments, a process improves the breast cancer detection capability of DM by testing additional miRNA biomarkers and by moving from purified oligos to total RNA from breast cancer cell lines and/or circulating miRNAs in human serum and plasma from cancer and normal patients. Since miRNAs are usually found within extracellular vesicles (EVs) the process preferably detects this fraction from serum. In some embodiments, results are benchmarked with those from RT-PCR.

In some embodiments, three human breast cancer cell lines are tested, MCF-7, MDA-MB-231 and SK-BR-3. These are available from ATCC and are well characterized for their miR expression levels. Additionally, they have been classified for their cancer subtype (either luminal or normal) and have expression patterns for clinically important protein biomarkers. These include estrogen receptor (ER), progesterone receptor (PR), epidermal growth factor receptor (EGFR), and ERBB receptor (ERBB). Their classification and phenotypes are as follows; MCF-7 (Luminal subtype, ER⁺, PR⁺, EGFR⁺, ERBB⁻); MDA-MB-231 (Normal-like, ER⁻, PR⁻, EGFR⁺, ERBB⁻); SK-BR-3 (Luminal subtype, ER⁻, PR⁻, EGFR⁻, ERBB⁺⁺). MDA-MB-231 cells are maintained in Leibovitz's L-15 Medium media supplemented with 10% FBS. MCF-7 and SK-BR-3 are maintained in Eagle's Minimum Essential Medium containing 0.01 mg/ml human insulin and 10% fetal bovine serum.

In some embodiments, peripheral blood is drawn from breast cancer patients under an approved protocol. In some embodiments, 20 mL peripheral blood is collected in Serum Separator Tubes (BD) for serum preparation. In some embodiments, tubes are incubated at room temperature for 30 minutes. Following clot formation, tubes can be centrifuged at 1,500 rcf for 10 minutes to separate the serum. For plasma preparation, 20 mL peripheral blood can be collected in K₂EDTA coated tubes (BD), centrifuged at 1,500 rcf for 10 minutes to separate the plasma and stored at −80° C. until further use. Serum and plasma samples can be stored in 1 mL aliquots at −80° C. Exosomes can be isolated using size exclusion chromatography by overlaying 1 mL of serum or plasma on qEV size exclusion columns (Izon) Exosomes can be eluted with PBS, filtered with an Ultrafree 0.22 μm centrifugal filter device (Millipore Sigma), and concentrated using an Ultra-4 10 kDa device (Amicon).

In some embodiments, frozen aliquots of serum and plasma from healthy donors and breast cancer patients are thawed on ice, and RNA is isolated using the miRNeasy Serum/Plasma Advanced Kit (Qiagen) according to manufacturer instructions. The RNA isolation kit utilizes a spin column-based protocol, requiring 200 μL of serum or plasma per spin column. Following isolation, the concentration of RNA can be quantified using a NanoDrop spectrophotometer. RNA purity can be verified by spectrophotometric ratios of A₂₆₀/A₂₈₀ of ˜2 and A₂₆₀/A₂₃₀ ratios greater than 2. RNA can be used for miRNA detection or stored at −80° C. for further use.

In some embodiments, total RNA isolation is carried out using approximately 5×10⁵ cells. Briefly, cells are lysed in RNAzol-BD reagent (Sigma-Aldrich) and cellular components (DNA, proteins, polysaccharides) precipitated using 1-bromo-3-chloropropane. RNA remains in solution and is carefully transferred to a fresh Eppendorf tube. The RNA is then precipitated from the aqueous phase with isopropanol, washed with ethanol and air dried. RNA can be quantified and stored as from peripheral blood.

In some embodiments, real-time quantitative polymerase chain reaction quantification of miRNA expression is performed using a TaqMan MicroRNA Assay kit (Applied Biosystems, Foster City, CA) according to the manufacturer's protocol. Total RNA is reverse transcribed using Multi-Scribe cDNA kit (Applied Biosystems). RT controls can be included in each set of reactions. PCR reactions are carried out in final volumes of 10 μL using an ABI 7900 HT Fast Real-Time PCR System (Applied Biosystems). Purified miRNA oligos can be used as a control and miR-16 used as an endogenous control to normalize miRNA expression. Threshold standard deviation (SD) for intra- and inter-assay replicates and PCR amplification efficiency can be calculated as described by the manufacturer.

In some embodiments, the sensors include 20 nm MNP, a 40 nm fluorescent NV-center nanodiamond and a pair of (partially) complementary DNA strands. The MNPs can be purchased from Ocean Nanotech (San Diego CA) and can have propargyl groups on the surface. The NV-center nanodiamonds have already been created and fully characterized. The FNDs can be coated with the biocompatible polymer glycidol. The glycidol-coated FNDs can be functionalized with two different chemical groups. Amine groups are attached through a 4-unit polyethylene glycol spacer (PEG₄) and a propargyl group is present through a PEG₁₂ spacer. The propargyl group is used for coupling DNA oligos having terminal azide groups via the well-known click chemistry reaction. The amine group is used to couple the biotin binding protein, streptavidin, to the nanodiamonds via standard NHS chemistry. Streptavidin is used to bind the sensor to biotinylated BSA-coated glass coverslips.

In some embodiments, Let-7a, miR-195 and miR-145 are chosen based on their previously documented associations with breast cancer, with miR-16 as an endogenous control for normalization. In some embodiments, the DNA version of the miR is conjugated to the nanoparticles rather than RNA, as they are much more stable. Single strand DNA may be purchased from the commercial supplier IDT. Single strand DNA conjugated to the FND may be fully complementary to the microRNA target and may be purchased with a poly dT tail and an azide group at the 5′-end. DNA conjugated to the MNP may correspond to a truncated miR sequence and may also have a polydT tail with a terminal azide at the 5′-end. The miR sensor may be formed from the MNP and FND conjugates as described in Example 6. The resulting sensors can have MNP and FND on opposite sides with a toehold region between them as shown in FIG. 13.

In some embodiments, miRNA concentrations in total RNA is quantified based on standard curves that plot average contrast polarization as a function of increasing concentrations of microRNA oligonucleotide. The standard curve can be created from ODMR contrast data for a sensor (e.g., miR-486) at known concentrations of a synthetic RNA oligonucleotide (e.g., miR-486). Uncoated FNDs can be run at the same time, and, from that data, a power function can be used to calculate a cutoff line. For a given concentration of oligo, the difference between the cutoff and each sensor data point can be determined and then averaged (with SD) for all the data points. This value is unitless and can be plotted as a function of oligonucleotide concentration. This should produce a straight line having an R² value of at least 0.95 and allows concentration to be determined directly from ODMR contrast. MicroRNA expression can also be calculated as fold change compared with an internal control miR-16, which is considered a stably expressed housekeeping microRNA.

If contrast changes in ODMR are not detected, the reaction can be enhanced by changing the NV-center density for fluorescence intensity, using a different conjugation chemistry, using another miR, coupling only a few oligonucleotides to the nanodiamond surface, using larger MNPs for their larger magnetic moments and thus larger contrast, or adjusting temperature, buffer, pH, or salt concentrations to enhance ODMR results.

In some embodiments, ODMR analysis is done one sample at a time using a custom-made holder for each coverslip, including positioning correctly over a 100× objective. Positioning slows ODMR time considerably (15 min per sample) but can be greatly improved by printing multiple samples on a single coverslip and performing just one positioning step before running the ODMR. This greatly simplifies sample analysis time, which would be a critical labor-saving step for cancer screening, detection, and staging. It is expected that it would take one to two minutes to perform ODMR on as many as 25 independent samples.

In some embodiments, a liquid handling system prints miR sensors as arrays in a 5×5 format on coverslips. Fluorescence can be used to characterize the array for uniformity, and the array can be validated using oligonucleotides. Specificity, sensitivity, and any cross contamination between spots can be evaluated. Finally, the array format can be used to determine miR expression patterns in plasma and serum prepared from breast cancer and normal peripheral blood samples.

Commercial liquid handling instruments are capable of printing arrays of any dimension and volume but are expensive, for example, $60,000 for Biodot's AD1520. In some embodiments, the arrays can be printed using a Prusa 13 MK2 3D printer integrated with a 0.5-10 μL 8-channel Integra Voyager Electronic Pipettor (Integra Biosciences, Hudson, NH). This pipettor has an adjustable pitch allowing array printing with variable pitch. In some embodiments, Labview, Python, or CAD software are used to integrate the printer with the pipettor. The printer/pipettor assembly can be operated in a dust-free, humidified glove box. The print area can be 250×210×200 mm, which allows the printing of several coverslips (20 mm×20 mm) at a time.

In some embodiments, arrays are printed by placing a glass coverslip adjacent to a 96-well PCR plate (0.2 mL/well) and using the pipettor to pull up sample volume to deliver 1 μL of sample on to the cover slip. The X and Y axis of the Prusa printer can be used for positioning the pipettor on the glass coverslip while the Z axis controls the height above the coverslip. Drop size can be controlled programmatically to ensure uniform spot sizes. With a pitch between 1 to 3 mm, and spot sizes of about 0.5 mm, a 5×5 array can be produced on a cover slip without overlap of spots.

The optical system can be extremely sensitive and can allow collection of meaningful fluorescence data with just a few μg of 40 nm NV-center nanodiamonds. This amount of fluorescent material can be printed and the array imaged later. Movement of the coverslip on the stage can be by an actuated stepper motor for coarse positioning in the X and Y direction and a fine-positioning piezo stage for raster scanning.

In some embodiments, characterization of the arrays is be done by printing a 5×5 array of streptavidin-FND conjugates (without DNA) on biotinylated cover slips. Imaging and quantifying relative NV-center fluorescence can confirm uniformity of spot size across the array and collect ODMR contrast for each spot of the array. There is preferably less than 5% variability of fluorescence spot intensity and ODMR contrast. Spot intensity is the median pixel intensity after background subtraction from the 532 nm laser. If needed, the spot size can be adjusted by adding glycerol to increase viscosity and/or PEG or ethanol to change surface tension.

Cross contamination between spots can be avoided by change tips or rinsing extensively between print steps. To determine cross contamination issues, a sample can be printed on the array, and then, without rinsing, several more spots of buffer can be repeatedly printed using the same pipette tip. Carryover can be checked by fluorescence imaging and ODMR.

In some embodiments, arrays for miR sensing are validated using an miR-486 sensor and synthetic RNA oligo. This sensor can then be transferred into 0.2 mL PCR tubes (96-well plate), and then either buffer or synthetic RNA oligo can be added to the tubes and strand displacement reactions can be allowed to occur. At the end of the incubation, the samples can be printed as a 5×5 array and then submitted to ODMR analysis. A buffer control (representing intact miR-486 sensor) produces the lowest contrast and can become the basis for calculating the cutoff line. miR-486 concentration can then be determined as described above. Unconjugated NV-center nanodiamonds, which should produce the highest contrast and be expected to produce nearly identical results to those shown in FIG. 13, can be run.

In some embodiments, miR sensors for sequences in Table 1 are created at a 5× to 10× larger scale so that there is enough for printing multiple coverslips. The sensors can be transferred into 0.2 mL PCR tubes (96-well plate) then either buffer or total RNA, and serum can be added to the tubes and strand displacement reactions allowed to occur. Samples can be run in triplicate using the controls discussed above. Additionally, a standard curve can be created for each miR using oligos for quantification as described above. At the end of the incubation the samples can be printed on an array and then submitted to ODMR analysis. Each coverslip preferably has only one type of miR. The advantage of this is that it should be easier to see differences among samples when they are all on the same coverslip. This also avoids potential problems with cross contamination from different miRs.

In some embodiments, statistical analyses are conducted. Generally, with limited number of replications in in vitro experiments, rank-based/permutation or re-sampling methods are not possible to provide accurate p-values. Instead, with few replications, appropriate transformations and the t-distribution can be used to generate p-values. For either continuous measurements under multiple treatment conditions or a series of repeated measures over multiple time points, linear mixed models, or non-linear mixed models when needed, can be used and the covariance structure can be estimated by the optimal unbiased method. All other experimental data can be analyzed via two sample t-tests or one-way analysis of variance (ANOVA) analyses, utilizing the Bonferroni method.

In some embodiments, the presence of environmental concerns, such as, for example, hazardous chemicals and viral and bacterial pathogens, is monitored. This permits detection and monitoring of threats such as bio- or chemical warfare agents, accidental contamination, persistent environmental contamination, and the effectiveness of decontamination processes.

Although the compositions, devices, and systems have been described for detection of a target material in a biological sample, the sample may alternatively be a non-biological sample.

In some embodiments, the presence of environmental concerns, such as, for example, hazardous chemicals and viral and bacterial pathogens, is monitored. This permits detection and monitoring of threats such as bio- or chemical warfare agents, accidental contamination, persistent environmental contamination, and the effectiveness of decontamination processes.

### EXAMPLES

The invention is further described in the context of the following examples which are presented by way of illustration, not of limitation.

### Nanodiamond Magnetometry System

Referring again to the drawings, a layout as schematically shown in FIG. 1 was built on a 6-ft×4-ft×12-in thick active-support table (Thorlabs Inc., Newton, NJ). A hanging shelf was installed above the table for the placement of electronic components. The optical design was for a confocal system having a pinhole to eliminate stray light and a galvanometer for scanning in the x-y plane. Additional components of this system included a 200-mW, 532-nm continuous wave diode-pumped solid-state (DPSS) laser representing the excitation source, and a 12-V acousto-optic modulator (AOM) that used sound waves to diffract and shift the frequency of light. The system included one iris and several lenses, mirrors, and filters, including a dichroic mirror and a 532-nm notch filter. The system used a 100× microscope objective (Mitutoyo Corporation, Kawasaki, Japan), a CCD camera, a photon counter, a microwave generator, a microwave analyzer, and a thin wire placed near the diamonds to create the microwave field. The last component of the optical system was a flow cell containing fluorescent nanodiamonds. The flow cell was fitted with a microwave wire and positioned above a 100× objective.

### Flow Cell

As schematically shown in FIG. 2, a flow cell assembly in an optical set-up for a nanodiamond magnetometry system included a polymeric chamber body approximately 3 cm wide, 3 cm long, and 5 mm deep with a sample inlet leading to a hollow test chamber into which the sample/buffer mixture flowed. Encircling the lip of the test chamber, which was flush with the surrounding surface of the flow cell body, was an indentation holding a rubber gasket, or O-ring, which kept the sample fluid confined to the sample chamber. At the edge of the flow cell was a raised lip, which created an indentation for holding a coverslip in place atop the surface of the cell. In the center of the test chamber was a ridge formed into the flow cell body and extending the width of the hollow chamber. The ridge was perpendicular to the flow of sample fluid, with a height that reached approximately 1 mm below the coverslip. This forced the sample fluid into close proximity with the coverslip, on the center of which an array of nanodiamonds was affixed. A heating pad was attached to the bottom of the flow cell and was used to increase the sample temperature for enhanced reaction rates. The sample fluid was prepared prior to injection into the chamber by controlled mixing of the test sample with a biologically relevant buffer such as phosphate buffered saline (PBS) at pH 7.5. This was accomplished by using two separate syringe pumps, one for the sample and the other for the buffer, to inject the liquids into a mixing chamber. After mixing, the sample/buffer mixture flowed through the sample inlet of the flow cell and into the test chamber.

### Relaxometry Measurements

T1 and T2 of NV centers were recorded on glass slides placed above a 10× objective lens of the optical set-up. T1 and T2 relaxation were determined by measuring fluorescence intensity as a function of time following initialization of the NV spin states with a 200-millisecond pulse using a 532-nm laser light source. Pulsed optical excitation from the 532-nm laser was controlled by an acousto-optic modulator (AOM), in combination with a PulseBlaster acquisition card. The photoluminescent emission was filtered by a 560-nm long pass filter in combination with a 650-750 nm bandpass filter and recorded using a single photon counting avalanche photodiode, and the time-correlated photons captured with a multiple-event time digitizer card. Data acquisition was controlled via custom LabVIEW code.

### ODMR Demonstration

The optical setup was modified to incorporate microwave field generation and magnets. Permanent magnets produced a magnetic field (B₀) of a few hundred gauss at a distance of a few centimeters from their surface and were preferrable over electromagnets. Testing included mixing a sample with buffer and flowing the mixture through a flow cell containing NV diamonds affixed to a glass coverslip. The NV diamonds on the coverslip were positioned over a 100× objective with surrounding magnet and microwave coils. In a typical ODMR measurement, the sample was illuminated with green light and the intensity of the red fluorescence was monitored while slowly sweeping an auxiliary microwave field into resonance. At resonance, a detectable reduction in fluorescence of 8% to 11% of fluorescence intensity was observed.

For ODMR studies, fluorescence at 637-nm was collected on an avalanche photo diode (APD) as the microwave frequency was swept from 2700 to 3000 MHz. The fluorescence intensity was plotted as a function of MW frequency, giving the ODMR spectrum. Since NV-centers are almost infinitely photostable, ODMR spectra were repetitively collectable without concern about quenching issues. The data was then averaged using statistical software and a zero-field resonance (dip) at 2,870 MHz (at zero magnetic field) was observed. The confocal setup used a single photon avalanche diode (SPAD) counter but alternatively could incorporate a high sensitivity Electron Multiplying CCD (EMCCD) camera, which can probe multiple individual nanodiamonds in parallel.

### Example 1

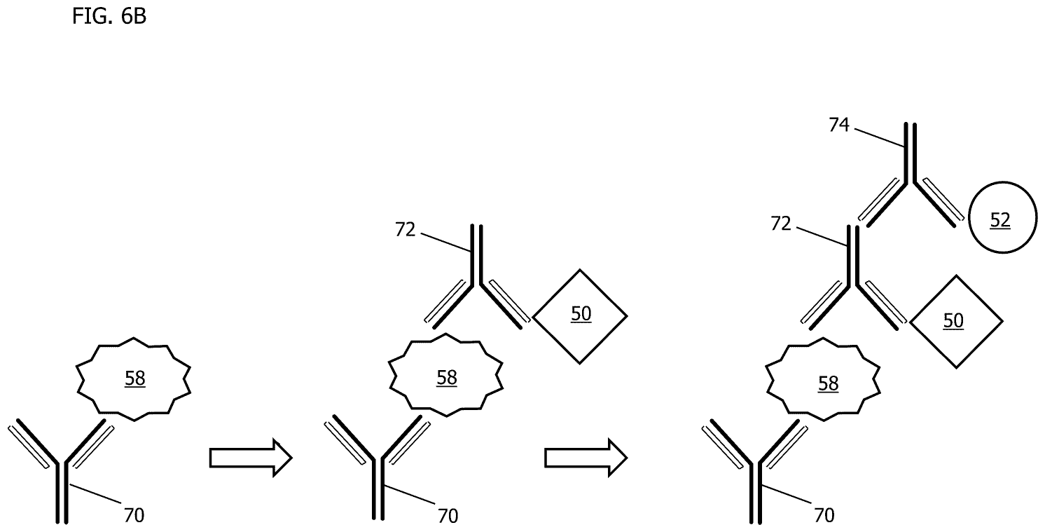

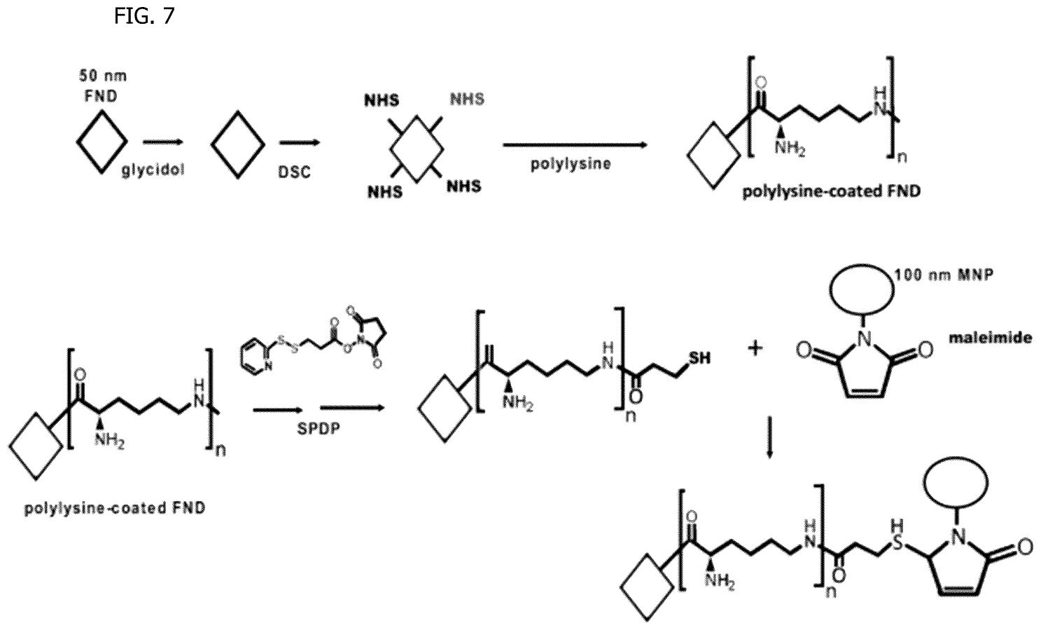

Referring again to the drawings, FIG. 7 shows a process for forming MND-probe conjugates. Glycerol, reagents, and organic solvents were obtained from MilliporeSigma (St. Louis, MO). Briefly, 20 mg of 40-nm fluorescent nanodiamonds were cleaned by refluxing for three days in a sulfuric acid:nitric acid (at a 9:1 ratio) solution. Twenty mg of cleaned nanodiamonds were resuspended in 2 mL of glycidol and placed in a 150-mL jacketed glass beaker. Approximately 60 mL of glycidol was added and the reactor was heated to 116° C. for 6 hours with continuous low power sonication (Misonix Sonicator 3000, Cole-Parmer, Vernon Hills, IL). Following cooling, unreacted glycidol was removed by extensive dialysis against 5 L of pure water for three days, with two complete exchanges each day. The nanodiamonds were centrifuged and the pellet suspended in pure water at 1 mg/mL. This coating step introduces reactive alcohol groups for subsequent conjugation steps.

Approximately one mg of glycidol-coated FND was suspended in a 1:1 mixture of dimethylacetamide (DMAC) and tetrahydrofuran (THF), centrifuged, and then resuspended in a mixture containing 0.1 mL of DMAC and 0.9 mL of dimethyl formamide (DMF). The nanodiamonds were activated with 0.2 mmole of N,N′-disuccinimidyl carbonate (DSC). After the final rinse in DMAC/THF solvent, the nanodiamonds were quickly resuspended in 10 mM 4-(2-hydroxyethyl)-1-piperazineethanesulfonic acid (HEPES)-0.01% Tween buffer, and 0.1 mL of a 0.1% polylysine solution was added. After shaking for 4 hours at room temperature, the aminated diamonds were rinsed and resuspended in DMAC/THF solvent.

The polylysine-coated FNDs had a high density of reactive amines on their surface. These were converted to reactive thiol groups by reaction with 2-iminothiolane (Traut's reagent). The imidoester group of the cyclic imidothioester reagent reacted with the amines to form a stable, charged linkage, having a sulfhydryl group. The product of this reaction, thiolated FNDs, was then reacted with a commercially available, 100-nm magnetic nanoparticle having reactive maleimide group on the surface. The terminal —SH group of the FND reacted with the maleimide group to create the final product, an MND-probe conjugate.

Referring again to the drawings, FIG. 8A shows the ODMR spectra for the thiolated FNDs. FIG. 8B shows the ODMR spectra for the MND-probe conjugates. Each graph shows time-averaged spectra for the FNDs under two different applied external fields. In both sets of graphs, a Zeeman shift is clearly observed for the FND. The Zeeman shift is much smaller under the weak magnetic field than the strong external magnetic field. However, the MND-probe conjugate shows hardly any resonance peaks and a clear absence in contrast can be seen in both magnetic fields. This demonstrates the change in contrast as well as a shift in the resonance peaks of the ODMR in the presence and absence of magnetic nanoparticles.

### Example 2

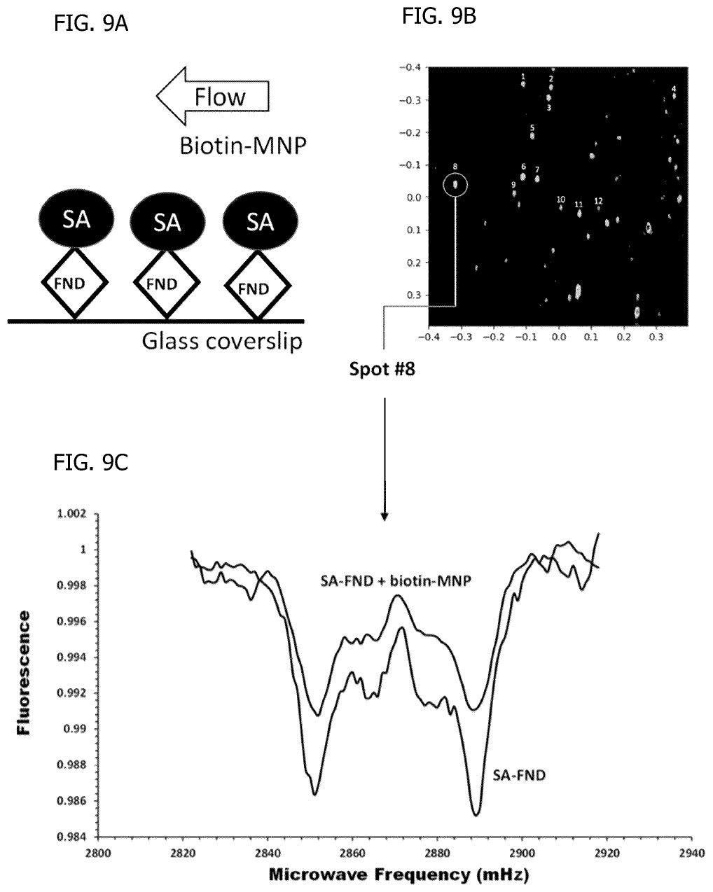

Referring again to the drawings, FIG. 9A shows a method of performing ODMR studies where the FND and MPs are attached to different target materials that come together through binding interactions. In this example, the binding interaction is between the biotin/streptavidin pair in which streptavidin is a protein that binds the small molecule biotin with extremely high affinity. Standard carbodiimide chemistry was used to link these proteins to FNDs and MPs. Streptavidin was conjugated to an NHS-FND to create a SA-FND conjugate. Amino-PEG-biotin (Broadpharm, San Diego, CA) was conjugated to a carboxylated MP using 1-Ethyl-3-(3-dimethylaminopropyl)carbodiimide (EDC) to create a biotin-MP.

Referring again to the drawings, FIG. 9A shows the overall design. Briefly, the SA-FNDs were spotted on an aminated glass coverslip then fitted to a flow cell. Phosphate-buffered saline was flowed across the flow cell using a syringe pump and ODMR spectra were collected. Next the biotin-MP was added to the PBS solution and allowed to flow across the SA-FND. The streptavidin recognized the biotin, thus bringing the MP close to the FND.

Referring again to the drawings, FIG. 9B shows a fluorescence heat map of the FNDs spotted onto the coverslip. The bright dots represent individual nanodiamonds, which allowed several spots to be chosen to monitor as the biotin-MP was flowed. In FIG. 9B, spot 8 was chosen to be followed. FIG. 9C shows the ODMR spectra for the SA-FNDs before and after the biotinylated-MP was passed across the flow cell. There was significant change in the NV-center contrast upon streptavidin binding the biotin-MP. This represents the expected results for using FNDs to detect the presence of a binding event between two molecules.

### Example 3

DNA-FND conjugates were formed using a 5′ or 3′ amine on the DNA. In these studies, a 50 base single-strand DNA was designed with limited, if any, secondary structure at room temperature. The oligonucleotide included a 6-carbon spacer and a terminal amine at the 5′ end. A short 17-mer single strand DNA strand complementary to the 50-mer was also purchased with the same amine group at the 5′ end.

The 50mer DNA strand was conjugated to glycidol coated FNDs via DSC activation as in Example 1. 50 μg of glycidol-coated FND was suspended in a 1:1 mixture of DMAC and THF, centrifuged and then resuspended in a mixture containing 0.1 mL of DMAC and 0.9 mL of DMF. The nanodiamonds were activated with 0.2 mmole of DSC by adding 50 mg DSC to the glycidol FND suspension and incubating for 4 hours. After the final rinse in DMAC/THF solvent, the nanodiamonds were quickly resuspended in PBS with 0.05% Tween-20, and 0.2 nanomoles of amine DNA 50mer was added. After shaking for 4 hours at room temperature, the diamonds were rinsed 3× in the same buffer.

The 17-mer DNA strand, complementary to the 50mer, was conjugated to a commercially available, 100 nm magnetic nanoparticle having a reactive maleimide group on the surface. The amine group on the cDNA was converted to reactive thiol groups by reaction with Traut's reagent. The terminal —SH group of the cDNA reacted with the maleimide group to create the final product, an MP-DNA composite. This was done as a one-pot reaction including 50 μg maleimide MPs in 200 μL PBS with 0.05% tween-20, 0.2 nanomoles of amine cDNA, and 2 nanomoles of Traut's reagent. After shaking for 2 hours at room temperature, the MPs were magnetically separated and rinsed 3× in the same buffer.

The detection of DNA is an important biomedical application of quantum-based sensing using FNDs. A 50 base DNA oligonucleotide was designed with an amine group at the 5′-end, as shown in FIG. 10. 40-nm FNDs having NHS group were prepared as described above and conjugated to the 5′ amine of the 50 base oligonucleotide (DNA-FND). A short complementary DNA sequence was coupled at the 5′ end to a 100-nm MP (DNA-MP). The DNA sequences are shown in the bottom panel of FIG. 10.

Referring again to the drawings, FIG. 10 shows ODMR results for the DNA-FND by itself and when it is hybridized with the DNA-MP. These results were collected in an optical setup having a biased magnetic field. Thus, both unhybridized and hybridized DNA-FNDs show a Zeeman shift. However, the ODMR spectrum for the hybridized DNA-FND shows broadening of the resonance peaks and this is different from that of the hybridized conjugate. These results demonstrate that simple hybridization of DNA can be detected by magnetometry.

### Example 4

Referring again to the drawings, FIG. 11 schematically shows a procedure for forming a stem-loop MND-probe conjugate. Approximately 50 μg of 40-nm FND was coated with glycidol then activated by DSC treatment as described above, washed, then resuspended in 200 μL PBS with 0.05% Tween-20 (PBST). Approximately 1 μL of 100 mg/mL amino propyl azide was added and the suspension was gently shaken for 4 hours at room temperature. The FNDs, now with azide groups on the surface, were then rinsed 3× in PBST and resuspended, and approximately 0.7 nanomoles of the functionalized stem-loop DNA was added. A copper-dependent click reaction was then performed to link the DNA to the azide on the nanodiamond surface. The reaction included tris((1-hydroxy-propyl-1H-1,2,3-triazol-4-yl)methyl)amine (THPTA), copper sulfate, and ascorbic acid at final concentrations of 1.25 mM, 0.25 mM, 0.84 mM, respectively. The sample reacted on a shaker overnight at 4° C. The DNA-FND conjugate was then rinsed 1× in PBST with 1 mM EDTA, then 2× PBST, then resuspended in 200 μL PBST. Commercially-available polymer coated MPs with carboxyl functional groups were used in the following steps. MPs were EDC-activated using 2-step EDC activations where 50 μg of the MPs were suspended in 200 μl PBST and 2 mg of EDC and 2 mg of NHS were added. The reaction proceeded for 15 min at room temperature to convert the carboxyl group to an amine reactive NHS ester. The activated MPs were then rinsed 2× PBST and resuspended in 200 μL PBST. The DNA-FND suspension was added to the MP suspension and reacted for 4 hours at room temperature. The resulting FND-DNA-MP, or MND-probe conjugate was then separated by magnetic separation and rinsed in PBST. The final product was then very gently resuspended in 500 μL PBST.

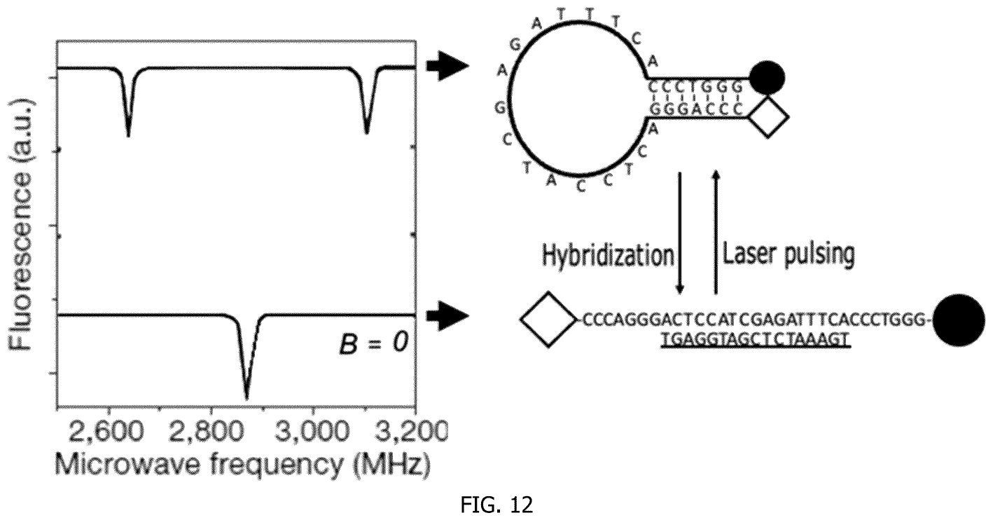

Referring again to the drawings, FIG. 12 shows the stem-loop DNA sequence that was designed to detect the single strand microRNA species, miR21. The DNA-FND composite was created in which an FND was attached at one end of the stem-loop sequence and a MP is attached at the other end of the DNA. Under room temperature conditions, base-pairing at the stem region brings the MP near the FND. This affects the fluorescence of the FND and leads to a Zeeman shift in the ODMR spectra as shown in FIG. 12.

The nanodiamonds are attached to a glass cover slip and then sealed within a flow cell. Fluid, containing single strand DNA or RNA, is then flowed through the flow cell with hybridization of complementary DNA/RNA occurring in the loop region. This destabilizes the stem region and results in a linear structure that moves the MP away from the FND. In the linearized configuration, the MP does not affect FND fluorescence, and a zero-field resonance peak is observed at 2.87 mHz. The linearized DNA can be returned to its original structure by a short series of laser pulses to rapidly denature the hybridized DNA. This approach is illustrated in FIG. 12.

### Example 5

Cleaned fluorescent nanodiamonds were coated with glycidol as described above. Approximately 1 mg of coated nanodiamonds are washed with a 50/50 mixture of N,N-dimethylacetamide (DMAC) and tetrahydrofuran (THF), then resuspended in N,N-dimethylformamide (DMF) containing 10% DMAC. Solid N,N′-disuccinimidyl carbonate (DSC) powder was added to 0.2 M DSC and vortexed, and the sample was allowed to react with shaking for 4-hours at room temperature. The activated gFND were then washed in DMAC and stored at −20° C. until use. Working quickly, NHS activated gFND were resuspended in 50 mM HEPES pH 7.4, 0.05% Tween-20 (HEPES-T) at a concentration of 10 mg/mL. Human IgG, rabbit IgG, goat anti-human IgG, or mouse IgG antibodies (Southern Biotech, Birmingham, AL) were quickly added to a final concentration of 1 mg/mL and incubated for 2-hours on an orbital shaker. Ethanolamine (10 μL) was added to quench the reaction and incubated for 10 minutes before rinsing three times with HEPES-T. The final volume was 1 mg/mL in the same buffer. Based on depletion studies, it was estimated that the antibody binding was between 1.5 to 3 ng IgG/mL per μg FND.

### Example 6

Chemicals and solvents were purchased from GFS Chemicals (Columbus, OH) and Sigma-Aldrich (St. Louis, MO). Antibodies were purchased from Southern Biotech (Birmingham, AL). The polyethylene glycol (PEG) linker, azido-PEG₁₂-NHS ester was purchased from Broadpharm (San Diego, CA). NHS-biotin was from Quanta Biodesign (Powell, OH), epichlorohydrin was from Alfa Aesar (Haverhill, MA), and N,N′-disuccinimidyl carbonate (DSC) was from Novabiochem (now Sigma-Aldrich). Glycidol was redistilled under reduced pressure at 31° C. prior to use.

FNDs were first coated with glycidol to introduce reactive alcohol groups for subsequent conjugation steps. Approximately one mg of glycidol-coated FND was suspended in a 1:1 mixture of dimethylacetamide and tetrahydrofuran (DMAC/THF), centrifuged and then resuspended in a mixture of DMAC and dimethyl formamide. The nanodiamonds were activated with 0.2 mmole of DSC then washed in DMAC/THF solvent. The nanodiamonds were quickly resuspended in HEPEST buffer and 0.18 mmoles of 4,7,10-trioxa-1,13-tridecanediamine was added. After shaking for 4 hours at room temperature, the aminated diamonds were rinsed and resuspended in DMAC/THF solvent.

Glycidyl propargyl ether (GPE) was synthesized by the reaction of propargyl alcohol with epichlorohydrin, in the presence of a strong base. Briefly, sodium hydroxide (NaOH) powder was mixed with dimethyl sulfoxide and approximately 0.6 mole of propargyl alcohol was added dropwise, while mixing and maintaining a temperature below 20° C. After mixing for 30 min, 1 mole epichlorohydrin was added dropwise and mixing continued for another 4 hours. The reaction was quenched by the slow addition of 300 mL of cold dH₂O, transferred to a separatory funnel and extracted five times using 30 mL of diethyl ether each time. The organic phases were combined in a round bottom flask and the diethyl ether driven off by a stream of dry nitrogen gas. The crude GPE was purified by vacuum distillation and the presence of the triple bond and epoxy group on the GPE was confirmed by FT-IR.

The propargyl group of GPE was introduced on the FND surface through the reaction of the epoxy group of GPE with the aminated FND. Limited reactivity between epoxides and aminated FNDs in 80 mM carbonate-bicarbonate buffer, pH 8.5 permitted the creation of aminated-propargyl FNDs having both amine and propargyl groups on the diamond surface.

Briefly, aminated FND were resuspended in 1 mL of an 80 mM carbonate-bicarbonate buffer, pH 8.5 and approximately 0.40 mmole of GPE was added. The solution was allowed to react overnight with vigorous shaking. The samples were then centrifuged and the nanodiamonds washed and resuspended with water. An assay for amine groups determined that approximately half of the amine groups remained on the FNDs following reaction with GPE in 80 mM carbonate-bicarbonate buffer pH 8.5.

Approximately 10 μg of anti-GR1 IgG antibody was diluted with 400 μL of 50 mM HEPES, pH 7.4, 0.05% Tween 20 buffer (HEPEST) and then reacted with a 10-fold molar excess of azido-PEG₁₂-NHS ester at room temperature for 2 hours. The reaction was transferred to a 10 k MWCO spin filter (Spin-X UF 500, Corning) and unconjugated PEG linker was removed by washing the filter with HEPEST. The azido-PEG₁₂-anti-GR1 conjugate was recovered from the spin filter by resuspension in 200 μL of HEPEST buffer. The concentration of the conjugate was estimated to be 50 μg/mL.

Conjugations to the aminated groups on the FNDs were completed before performing the click reactions. Approximately 0.2 mmole of DSC was incubated with 1 mg of aminated FNDs in a one mL volume at room temperature for 4 hours. The FNDs were rinsed thrice with DMAC/THF before quickly resuspending in HEPEST buffer and adding 2 μg of anti-CD11b. The mixture was incubated for 2 hours at room temperature after which the resulting diamond immunoconjugate was centrifuged, resuspended in 500 μL of HEPEST and then divided into five-100 μL aliquots.

In a related set of reactions, NHS-PEG₁₂-biotin was conjugated instead of anti-CD11b antibodies to the aminated-propargyl-FNDs. In these experiments DSC activation was not conducted. Briefly, one mg of aminated-propargyl-FNDs was mixed with 10 μL of 1 mg/ml NHS-PEG₁₂-Biotin and allowed to react for 2 hours. The biotinylated FND was rinsed thrice, resuspended in HEPEST and divided into five-100 μL aliquots.

Click reactions were performed on both FND-immunoconjugates and biotinylated FNDs. Approximately 2 μg of azide-modified anti-GR1 IgG antibody was added to each tube containing 100 μL of FND and allowed to react for 5 min. Approximately 50 μL of 20 mg/mL (46 mM) tris-hydroxypropyl-triazolylmethylamine (THPTA) was mixed with 25 μl of 5 mg/mL (31.3 mM) copper sulfate and allowed to react for 5 minutes. Fifteen microliters of the THPTA/CuSO₄ mixture was added to each tube of FND solution, and quickly followed with 12 μL of 5 mg/mL (25.2 mM) sodium 1-ascorbate. The resulting mixture was incubated 24 hours at 4° C. with vigorous shaking, centrifuged and resuspended in HEPEST buffer containing 1 mM ethylenediaminetetraacetic acid (EDTA).

In one set of studies, only anti-GR1 IgG antibodies were conjugated to the aminated-propargyl-FNDs. This immunoconjugate was created by first reacting 1 mg of aminated-propargyl-FND with 10 μL of 1 mg/mL m-dPEG₂-NHS ester for 2 hours to block available surface amines. The anti-GR1 IgG antibody was attached as described above.

Succinimidyl 3-(2-pyridyldithio)propionate (SPDP), 20 mM in DMAC, was used to detect amines on the surface of FND. Briefly, aminated FNDs were suspended in 1 mL phosphate buffered saline containing 1 mM EDTA (PBS-EDTA) and 25 μL of SPDP solution was added. Following a two hour incubation on a shaker at room temperature, the mixture was centrifuged and the FND pellet rinsed and resuspended in PBS-EDTA. Ten microliters of 15 mg/mL dithiothreitol (DTT) was added and samples mixed on shaker for 15 min. The DTT reducing agent cleaves pyridine-2-thione from the SPDP-nanodiamond adduct, which can be separated from the nanodiamonds by centrifugation at 20800 relative centrifugal force (rcf). Triplicate 200 μL aliquots of supernatant were put in a 96-well clear polystyrene plate and the absorbance at 350 nm was read using a Tecan Genios Plus microplate reader. A standard curve was created by making serial dilutions of 20 mM SPDP solution in PBS-EDATA which were then reduced with DTT and absorbance read at 350 nm.

A 10 μg/mL solution of Streptavidin was prepared in 200 mM carbonate buffer pH 9.4, and used to coat the wells of a 96-well polystyrene plate (Nunc) overnight at 4° C. The wells were then washed extensively with 50 mM HEPES pH 7.4, 0.05% Tween 20, (rinse buffer) then blocked for 2 hours with 300 μL of 20 mg/mL bovine serum albumin in the same buffer at room temperature.

FNDs were diluted to 100 μg/mL in rinse buffer containing 10 mg/mL BSA, and 100 ul of each FND type added to each well. The plate was allowed to incubate for 2 hours at room temperature with gentle shaking. Each well was then gently washed thrice with rinse buffer then incubated with 100 μL of goat anti-rat-HRP (diluted 1000× in rinse buffer). The plate was allowed to react for 2 hours at room temperature with gentle shaking then each well was gently rinsed thrice with rinse buffer. The wells were developed with 100 ul of a 3,3′,5,5′-tetramethylbenzidine (TMB) solution and after 5 minutes the reaction quenched by the addition of 100 μL of 1M H₂SO₄. The absorbance in each well was read at 450 nm in a Tecan Genios Plus plate reader (Tecan Trading AG, Maennedorf, Switzerland).

### Example 7

The miRNAs miR-486, miR-421, miR720, miR-1303, and miR-503 have been reported to be predictive of inflammatory breast cancer. MiR-21 is frequently upregulated in several cancers. Single strand DNA representing the complementary sequence of these microRNAs were synthesized by a commercial supplier with a poly dT tail and an azide group at the 5′-end. The oligos were then coupled to 40 nm FNDs through a copper-catalyzed click reaction between the azide and propargyl groups. The amine group on the nanodiamond was converted to an NHS ester using the zero-length carbodiimide, N,N′-disuccinimidyl carbonate (DSC), which was then conjugated to streptavidin. A second single strand DNA corresponding to a shortened version of the microRNAs was synthesized with a poly dT tail and a propargyl group at the 5′ end. This DNA was coupled to an azide-modified 15 nm MNP purchased from Ocean Nanotech and purified by centrifugation.

The DNA sequence on the nanodiamond was fully complementary to the microRNA target, while the DNA sequence on the MNP was a shortened sequence of the microRNA. This created a toehold, which is a region of single-stranded DNA that allows an invading strand to bind and initiate strand displacement. This is shown schematically in FIG. 13 and shows a toehold region that allows the microRNA (miR-486) to displace the DNA-MNP conjugate. Release of the DNA-MNP conjugate moves the MNP away from the FND and restores the ODMR contrast.

An FND-MNP nanocomposite, hereafter “miR-sensor,” was formed by mixing the FND and MNP conjugates in a 1:1 mass:mass ratio at room temperature and allowing the complementary strands to hybridize. After 3 hours, the newly formed miR-486 sensor was divided in half and mixed with either buffer or with 1 nmole of synthetic mir-486 RNA. After thirty minutes, the samples were drop-cast onto glass coverslips that were coated with biotinylated BSA. The streptavidin on the FNDs bound the biotin groups and were thus retained on the glass slide. The coverslips were washed then submitted to ODMR. 1 μL of a 10-pM solution of the mir-486 sensor, essentially a few femtograms of sensor, was used, due to the extreme sensitivity of DM and confirmed that independent fluorescent spots were non-overlapping by imaging.

A portable optical system was built for recording ODMR spectra and its use for ODMR studies was validated through federal contracts from the NCI and the Department of Defense. The sensors were on a coverslip above a 100× objective, the sensors were excited with a 532 nm laser, and the fluorescence above 650 nm was recorded using a long pass filter. Rather than sweeping the microwave frequency from 2.7 to 3 GHz, fluorescence was acquired with the microwave field off and with applied microwave fields of 2.864, 2.865 and 2.866 GHz. The output from the three microwave fields was averaged and compared to the fluorescence output in the absence of the microwave field. The ratio of photon counts in the presence and absence of the microwave field is termed ODMR contrast.

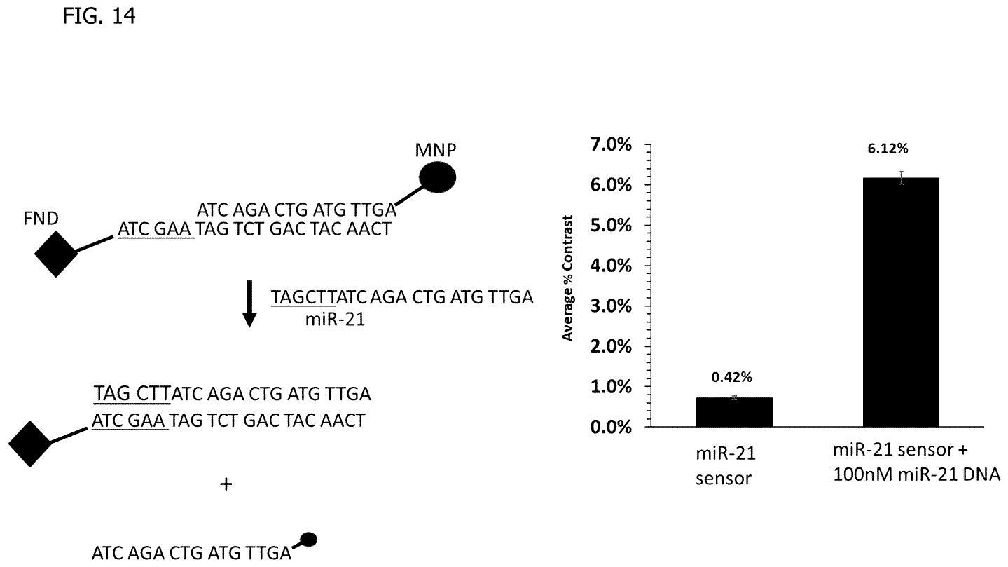

FIG. 13 shows a schematic of how DM processes are performed using magnetic nanoparticles (black circle) and fluorescent NV center nanodiamond (diamond) that are linked together through a short duplex DNA. The toehold region is shown. FIG. 14 shows polarization results for uncoated nanodiamonds (gray dots) which have higher contrast than miR-486 sensor (black dots), due to the MNP. After the addition of 1 nM miR-486, the contrast rises about the cutoff line (dashed line).

FIG. 14 shows representative results from an ODMR process. The data is plotted as a log-log plot with each dot representing contrast results from individual FND particles. There is an exponential relationship between overall contrast and photon counts, which fits a power function having an R² value of 0.99 for uncoated FNDs. The power function equation (y=a*x^{b}) was used for the “buffer only” sample (i.e., mir-486 sensor alone) to calculate the upper limits of the miR-486 sensor and the calculate line served as a cutoff value. The dots above the cutoff line are considered to represent nanodiamonds not containing MNPs, whereas below this line the nanodiamonds are considered to contain an MNP.

The overall contrast from the 40 nm FNDs in FIG. 14 is highly reproducible and in the presence of a paramagnetic nanoparticle (i.e., buffer only), the polarization drops below the calculated cutoff line. Addition of 1 nM miR-486 RNA oligonucleotide leads to hybridization at the toehold region and subsequent release of the MNP. The FND contrast was thereby restored to that of the uncoated FND (lacking the MNP).

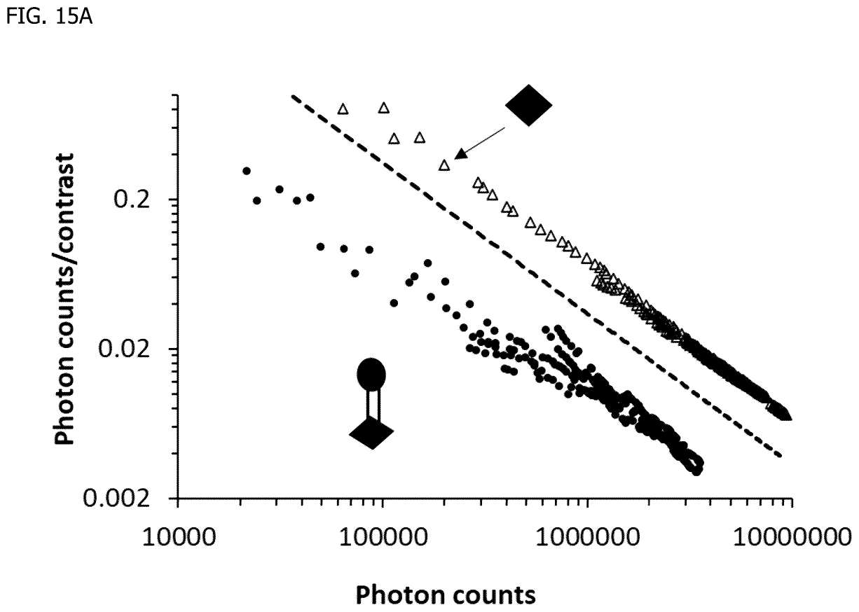

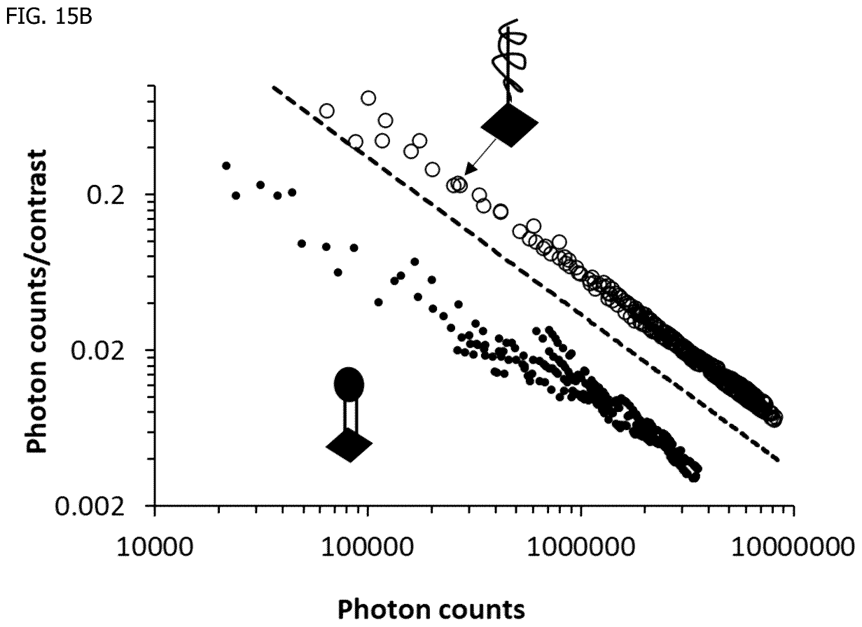

FIG. 15 shows the specificity of the miR-21 sensor towards other miRNAs. The graph shows the percentage of the miR-21 sensor that released the MNP when 1 nM of miR-21, miR-1303, miR-421, or miR-486 oligos were added. The sensor did not release the MNP until miR-21 oligo was added. The miR-21 sensor did not react with other miR oligos. A miR-21 RNA oligo was able to cause the release of the MNP which restored the contrast to that of the uncoated FND. However, other miRNA oligos had no effect on the overall contrast of the miR-21 sensor with the data points remaining below the calculated cutoff. Serial dilution studies with the mir-486 sensor were performed in triplicate and showed that the miR-486 sensor could detect between 0.1 to 1 pmole of miR-486 RNA oligo.

The results suggest the use of diamond magnetometry for cancer diagnosis, cancer monitoring and response to therapy. With additional details and demonstrations, such as, for example, demonstrating DM with total RNA preparations, quantifying microRNAs by DM, and developing an array format to analyze multiple microRNAs, the technology may move into a research or clinical setting.

### Example 8

FIG. 16 shows an alternative method of analyzing the raw data of Example 7. In this approach, the raw data is represented in a histogram showing the distribution of ODMR contrast of each individual FND. Each dataset includes the ODMR response of 50-2000 individual FNDs. The distribution of the sensor (MNP hybridized to the FND) can be compared to the distribution of the positive sample (MNP displaced from FND). Multiple statistical methods can be applied to the two datasets in order to determine the difference and confidence interval. These statistical methods may include, but are not limited to, the mean, the median, the Kolmogorov-Smirnov test, and the Mann-Whitney U test. The average of each dataset is represented in the bar graph. The error bars in the bar graph represent the repeatability of the average contrast. Three to five different scans of each sample are preformed and the average ODMR contrast is determined for each scan. The standard deviation is then calculated from each individual ODMR average. The datasets in the histogram are aggregates of all the individual scans.

### Example 9

An aptamer-based sensing approach may be used as an alternative to the stem-loop and toehold DNA displacement sensing approaches described herein. An aptamer is a synthetic DNA, RNA, xeno nucleic acid (XNA), or peptide that selectively binds to a specific target molecule. The aptamer can be engineered through Systematic Evolution of Ligands by EXponential enrichment (SELEX) technology to have a range of binding affinities to the target molecule. Aptamers have secondary structures that can undergo conformational switching upon ligand binding. In this Example, the aptamer is designed to bind the small molecule cortisol and subsequently undergo a structural change.

This Example takes advantage of the conformational transition by creating a magnetic nanoparticle with a complementary sequence to a short region of the aptamer. Upon cortisol binding, this MNP-DNA construct is displaced from the aptamer. One such aptamer is shown in FIG. 17. FIG. 17 shows a sensor where the aptamer is conjugated to the FND and the MNP-DNA (A-probe) is hybridized to the aptamer sequence. This sensor has a magnetic nanoparticle close to the FND and subsequently shows a low level of contrast as determined by magnetometry. Cortisol binding causes a conformational change or switching on the aptamer, which displaces the MNP-DNA strand and releases the MNP. The resulting FND is not affected any longer by the MNP and its contrast rises by as much as several percent. The average contrast is shown as a bar graph in FIG. 17, which can be easily interpreted by someone who is not an expert in magnetometry.

### Example 10

Different fluorescent nitrogen-vacancy nanodiamond-probe-reporter conjugates and strategies may be employed for nucleic acid detection.

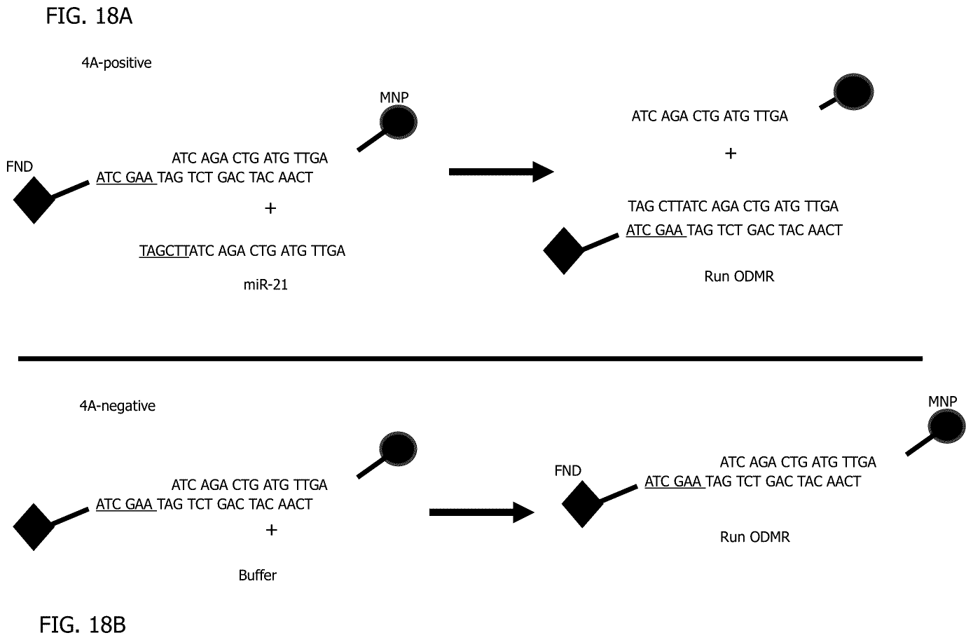

FIG. 18A and FIG. 18B show a first conjugate and strategy, where the conjugate is created and is then exposed to a mixture containing or potentially containing the target material. In this instance, a positive, as shown in FIG. 18A, results in the target releasing the magnetic nanoparticle and returns the contrast to that of an uncoated FND. A negative, as shown in FIG. 18B, leads to no change in the conjugate.

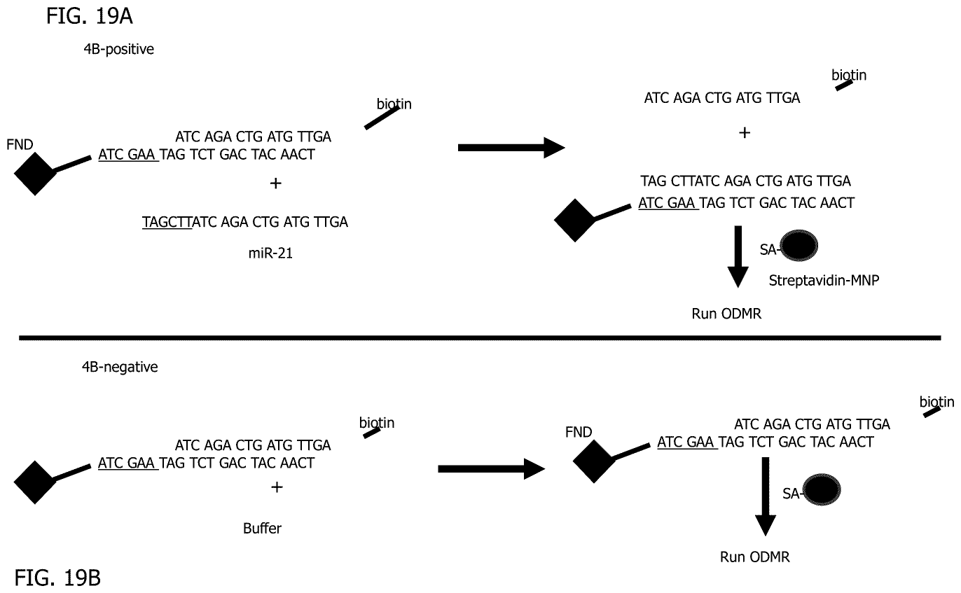

FIG. 19A and FIG. 19B show a second conjugate and strategy. In this case, a biotin group replaces the magnetic nanoparticle and after the reaction with the target, the biotin tag is removed if there is a positive response, as shown in FIG. 19A. The result for a positive response is essentially no change in contrast. For the negative or control, as shown in FIG. 19B, the biotin group remains hybridized and upon addition of an MNP-avidin or MNP-streptavidin complex, the MNP-avidin or MNP-streptavidin complex binds to the biotin on the sensor and this causes a reduction in contrast. As with the first approach, these reactions require a comparison between two groups.

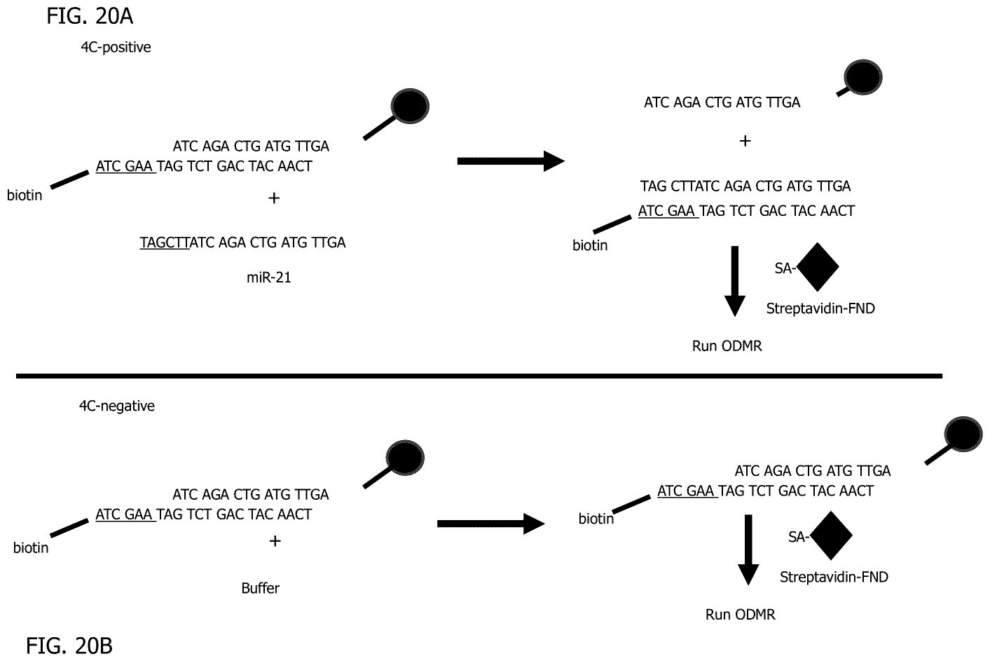

FIG. 20A and FIG. 20B show a third conjugate and strategy. This is similar to the first conjugate and strategy, except that biotin tag replaces the FND on the conjugate. After exposure to the sample, an FND-avidin or FND-streptavidin complex is added. In the positive response of FIG. 20A, no coupling occurs. In the negative response of FIG. 20B, coupling does occur.

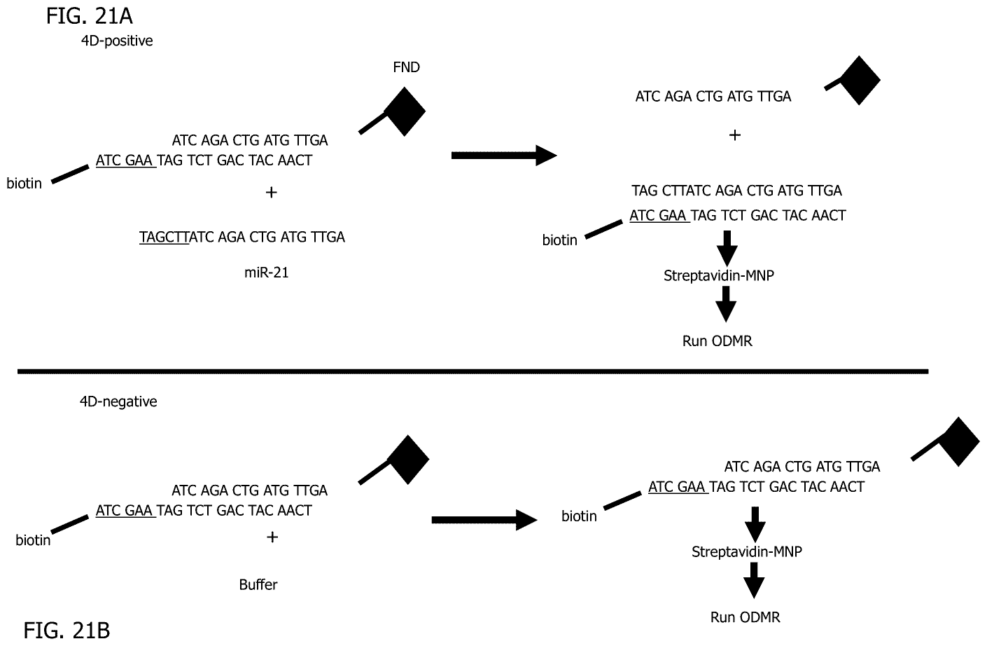

FIG. 21A and FIG. 21B show a fourth conjugate and strategy. This is similar to the second conjugate and strategy, except that biotin tag and the FND have switched places on the conjugate. After exposure to the sample, an MNP-avidin or MNP-streptavidin complex is added. In the positive response of FIG. 21A, no coupling occurs. In the negative response of FIG. 21B, coupling does occur.

The foregoing are representative embodiments of a sensing system according to the disclosure. Importantly, in the operation of the system the coupling and/or release of a magnetic nanoparticle to and from a construct that includes an NV-center nanodiamond, where the construct includes a biomolecule that has binding affinity for a target, permits actuation of the sensor.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A system for detecting a target material, the system comprising:
a plurality of fluorescent nitrogen-vacancy nanodiamond-probe-reporter conjugates (MND-probe conjugates), each MND-probe conjugate comprising:
at least one fluorescent nitrogen-vacancy nanodiamond (FND);
at least one reporter selected from the group consisting of a magnetic particle (MP), a paramagnetic complex, and a chemical; and
at least one polymeric probe comprising a biomolecule selected from the group consisting of at least one polynucleotide, at least one polypeptide, and a combination thereof, wherein the polymeric probe is characterized as having binding specificity to the target material;
wherein each of the at least one FND and the at least one reporter is linked to the at least one polymeric probe, and
wherein a distance between the FND and the reporter in the MND-probe conjugate changes when the at least one polymeric probe binds to the target material and displaces or releases the target material.

2. The system of claim 1, wherein the at least one reporter is selected from the group consisting of a nanoscale MP, a paramagnetic complex, a gadolinium complex, and a chemical.

3. The system of claim 2, wherein the at least one reporter is a chemical comprising biotin, the system also comprising a magnetic particle comprising avidin or streptavadin.

4. The system of claim 1, wherein the biomolecule is at least one polynucleotide, the at least one polynucleotide being selected from the group consisting of duplex deoxyribonucleic acid (DNA) comprising a first DNA strand and a second DNA strand, at least a portion of the second DNA strand being complementary with at least a portion of the first DNA strand; a pair of polynucleotide strands comprising a first strand and an aptamer strand, the aptamer strand being complementary to at least a portion of the first strand; and a single polynucleotide strand comprising two regions of complementarity.

5. The system according to claim 1, wherein each of the plurality of MND-probe conjugates includes one FND, one reporter, and one polymeric probe.

6. The system according to claim 1, wherein the target material is selected from the group consisting of (i) a small ribonucleic acid (RNA) of less than 200 bases comprising a microRNA (miR), (ii) a small ribonucleic acid (RNA) of less than 200 bases comprising a small noncoding RNA (sncRNA), (iii) a small ribonucleic acid (RNA) of less than 200 bases comprising a transfer RNA (tRNA), (iv) a small ribonucleic acid (RNA) of less than 200 bases comprising a short interfering RNA (siRNA), (v) a deoxyribonucleic acid duplex, (vi) a peptide, (vii) a small molecule, and (viii) combinations thereof.

7. The system according to claim 1, wherein:
the polymeric probe comprises a nucleic acid duplex comprising a first nucleic acid strand coupled to the reporter and a second nucleic acid strand coupled to the FND, the second nucleic acid strand being at least partially complementary to the first nucleic strand, each of the first and second strands having a first end and a second end corresponding to a first end and a second end of the polymeric probe;
at least one of the nucleic acid strands has binding affinity for the target material;
the first nucleic acid strand is four to eight bases shorter than the second nucleic acid strand;
the second nucleic acids comprise four to eight bases that are not complementary to the first nucleic acid and form a toe hold region when the first and second nucleic acid strands are hybridized; and
each of the reporter and the FND are coupled to the respective first and second nucleic acid strands at the same end of the MND-probe conjugate or at opposite ends of the MND-probe conjugate.

8. The system according to claim 1, wherein the at least one FND is coated with a first plurality of chemical functional group linkers comprising glycidols and the at least one reporter is coated with a second plurality of chemical functional group linkers comprising carboxyl moieties to chemically couple the FND to the reporter, and wherein the at least one FND has a particle size in the range of about 10 nm to about 1000 and the at least one reporter has a magnetic core size in the range of about 5 nm to about 100 nm.

9. The system according to claim 1, wherein the polymeric probe is characterized as having binding specificity to the target material, wherein an FND-probe conjugate binds to the target material, and wherein a distance between the FND and the reporter in the MND-probe conjugate changes when the FND-probe conjugate binds to the target material.

10. The system according to claim 1, wherein the polymeric probe is characterized as having binding specificity to the target material, wherein a reporter-probe conjugate binds to the target material, and wherein a distance between the FND and the reporter in the MND-probe conjugate changes when the reporter-probe conjugate binds to the target material.

11. A process for detecting the presence of a target material in a sample, the process comprising:
providing the plurality of MND-probe conjugates according to claim 1;
providing the sample;
providing an interrogation system capable of detecting at least one measurable change relating to the displacement of the reporter toward or away from the FND;
introducing the plurality of MND-probe conjugates into the detection system in contact with the sample suspected of containing the target material; and
interrogating the MND-probe conjugates-sample combination to detect a measurable change.

12. The process of claim 11, wherein the measurable change is a change in fluorescence.

13. The process of claim 11, further comprising providing a flow cell and a fluid medium for receiving the sample, the sample comprising a biological sample from one of cell, tissue, or a combination thereof, wherein one or more of the plurality of MND-probe conjugates are immobilized on a transparent substrate of the flow cell and the flow cell is configured to flow the sample over the immobilized reagents.

14. The process of claim 11, wherein the transparent substrate is selected from the group consisting of a glass slide, a glass plate, an array titration tray, and a clear polymeric substrate.

15. The process of claim 11, further comprising providing a transparent substrate and a fluid medium for receiving the sample, the sample comprising a biological sample from one of cell, tissue, or a combination thereof, wherein one or more of the plurality of MND-probe conjugates are immobilized by drop casting on the transparent substrate with an immobilizing agent sufficient to provide binding capture for a diamond concentration in the range of about 0.1 ng/L to about 1000 ng/mL and a time period in the range of about 10 seconds to about 10 hours such hat the distribution of nanodiamonds is a punctate single layer across the substrate surface.

16. The process of claim 11, the interrogation system comprising optics configured to generate and measure the polarization of an NV-center of the FND or one or more of optically detected magnetic resonance (ODMR), a spin-lattice relaxation time (T1), a spin-spin relaxation time (T2) of the plurality of magnetic particles, or a combination thereof.

17. The process of claim 16, wherein measuring ODMR of the plurality of fluorescent nitrogen-vacancy nanodiamonds after exposure to the biological sample determines a presence or an absence of the target material in the biological sample based on the polarization, the ODMR, the T1, or the T2, and wherein measuring a spin-lattice relaxation time (T1) or a spin-spin relaxation time (T2) of the plurality of MND-probe conjugates after exposure to the biological sample determines a presence or an absence of the target material in the biological sample based on values of T1 or T2.

18. The process of claim 11, wherein the process is used in a breast cancer screening.

19. A reagent for detection of a target material, the reagent comprising:
a plurality of fluorescent nitrogen-vacancy nanodiamond-probe-reporter conjugates (MND-probe conjugates), each MND-probe conjugate comprising:
at least one fluorescent nitrogen-vacancy nanodiamond (FND);
at least one reporter comprising a magnetic nanoparticle particle (MNP); and
at least one polymeric probe, the polymeric probe comprising a first nucleic acid strand coupled to the MP and a second nucleic acid strand coupled to the FND, the first nucleic acid strand being at least partially complementary to the second nucleic acid strand to releasably couple the MP to the FND via the interaction between the complementary first and second nucleic acid strands, one of the nucleic acid strands having binding affinity for the target material;
wherein a distance between the FND and the reporter in the MND-probe conjugate changes when the at least one polymeric probe binds to the target material and displaces or releases the target material.

20. The reagent of claim 19, wherein the target material is cortisol and the cortisol causes a conformational change in the polymeric probe, which produces positional changes in a spatial relationship of the MNP and the FND in the MND-probe conjugate, or wherein the target material is a microribonucleic acid biomarker of breast cancer and the polymeric probe comprises a deoxyribonucleic acid having a single-stranded toehold portion for the target material to bind and initiate strand displacement of the polymeric probe.
